(19) United States
(12) Patent Application Publication
(10) Pub. No.: US 2004/0142831 A1
(43) Pub. Date: Jul. 22, 2004

(54) Cosmetic composition comprising rigid fibres and at least one compound chosen from film-forming polymers and waxes

(76) Inventor: Nathalie Jager Lezer, Verrieres-Ie-Buisson (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/665,007

(22) Filed: Sep. 22, 2003

Related U.S. Application Data
(60) Provisional application No. 60/416,235, filed on Oct. 7, 2002.
(60) Provisional application No. 60/416,238, filed on Oct. 7, 2002.

(30) Foreign Application Priority Data
Sep. 20, 2002 (FR) 02 11646
Sep. 20, 2002 (FR) 02 11647

(51) Int. Cl. A61K 7/06; A61K 7/50

(52) U.S. Cl. 510/130; 424/70.1

(57) ABSTRACT

Disclosed herein is a cosmetic composition comprising substantially rectilinear rigid fibres and at least one compound chosen from film-forming polymers and waxes, the composition having a thixotropic plastic behaviour.

The composition exhibits a good dispersion of the rigid fibres and allows for a homogeneous deposition on the keratinous materials. For example, the composition can confer a good effect of lengthening of the eyelashes.

Also disclosed herein is a method for making up and care of keratinous materials, for instance applying the composition as a mascara.

This application claims benefit of U.S. Provisional Application No. 60/416,235, filed Oct. 7, 2002, and U.S. Provisional Application No. 60/416,238, filed Oct. 7, 2002.

Disclosed herein is a composition comprising, in a physiologically acceptable medium, rigid fibres and at least one compound chosen from film-forming polymers and waxes, intended for, for example, cosmetic applications. Also disclosed herein is a method for making up or for the cosmetic care of keratinous materials using this composition. The composition and the method according to the invention may be, for instance, intended for keratinous materials such as the skin, including the lips, and superficial body growths such as the eyelashes, the eyebrows, the hair and the nails, of, for example, human beings. The disclosure also relates to a mascara.

The composition according to the disclosure may be provided in the form of a product for coating keratinous fibres such as the eyebrows, the hair, the eyelashes, and for instance, in the form of a composition for coating the eyelashes, such as a mascara, an eyeliner, a product for the lips, a blusher or an eye shadow, a foundation, a make-up product for the body, a concealer, a nail varnish, or a care product for the skin.

The composition for coating keratinous fibres may be a make-up composition, a composition to be applied over a make-up, also called top coat, or alternatively a composition for the treatment (or care) of the eyelashes, the eyebrows or the hair.

It is known, in this field of the art, to use fibres in make-up or care compositions for keratinous materials for improving their cosmetic properties.

For example, it is known from the document JP-A-3 151 613 to use fibres in mascara compositions for conferring a lengthening and thickening effect on the eyelashes. The documents JP-A-57 158 714 and JP-A-9 263 518 describe mascara compositions comprising fibres and polymers of the acrylic type in aqueous dispersion.

Likewise, the documents JP-A-6 9340 and JP-A-7 179 323 describe mascara compositions comprising fibres, for example, nylon fibres, and film-forming polymers in aqueous dispersion.

The document FR-A-2 817 477 describes cosmetic compositions or formulations comprising fibres based on a synthetic or artificial polymer, such as polypropylene, PET, polyamide 6 and polyamide 66.

Compositions comprising fibres generally comprise a thickening agent in order to provide the composition with a consistency which allows easy application of the composition to the keratinous materials. However, it has been observed that some thickeners such as hydroxyethylcellulose do not make it possible to obtain a good dispersion of the fibres in the composition: the fibres may be poorly distributed during the application of the composition to the keratinous materials, the fibres may be therefore randomly oriented and distributed on the keratinous materials, and this may result in a heterogeneous deposit which damages the expected good cosmetic properties. For example, for a mascara, the poor dispersion of the fibres can lead to a mediocre and non-homogeneous lengthening effect.

For instance, mascara compositions comprising fibres may not make it possible to obtain an optimum and long-lasting lengthening effect because this effect is rapidly lost after the application of the composition. Indeed, the fibres may not be situated in the extension of the eyelashes.

The effect obtained by the mascara compositions containing prior art fibres is often aesthetically unacceptable, for instance in the case of bushy and/or long and/or curly eyelashes for which an unsightly so-called “Christmas tree” appearance of the eyelashes may be obtained.

A need therefore exists for a cosmetic composition, such as a mascara composition, which makes it possible to obtain excellent cosmetic properties, and a homogeneous and precise make-up application.

A need exists also for a mascara composition that offers a lengthening, in a perfect continuity of the eyelash, a non-random, regular positioning of the fibres exactly in the continuation of the eyelash and therefore an optimum aesthetic effect. The composition should furthermore adhere well to the keratinous fibres, for example to the eyelashes, should not form lumps and should be easy and quick to apply.

One aim of the present disclosure is, inter alia, to respond to the needs and to satisfy the requirements mentioned above.

It has been discovered that by using particular fibres in a composition comprising at least one compound chosen from film-forming polymers and waxes, and the composition having a thixotropic plastic behaviour, a composition is obtained which comprises a homogeneous dispersion of fibres and which therefore can lead to a homogeneous and precise making up of the keratinous materials, such as the eyelashes. For example, when the composition is a mascara, the fibres applied to the eyelashes become oriented and become fixed in the continuation of the eyelashes, regardless of the typology of the eyelashes. The fibres are not therefore randomly arranged, but become placed in the continuation of the eyelashes.

For example, one aspect of the disclosure is a composition comprising, in a physiologically acceptable medium, substantially rectilinear rigid fibres and at least one compound chosen from film-forming polymers and waxes, the composition having a thixotropic plastic behaviour.

Another aspect of the invention is a cosmetic method for making up and caring for keratinous materials, such as the eyelashes, comprising the application to the keratinous materials of a composition as defined above.

Another aspect of the disclosure is the use of a composition as defined above in order to obtain a homogeneous deposit, such as a make-up, on the keratinous materials.

Still another aspect of the disclosure is the use of substantially rectilinear, rigid fibres in a composition comprising, in a physiologically acceptable medium, at least one compound chosen from film-forming polymers and waxes, the composition having a thixotropic plastic behaviour, in order to obtain a homogeneous deposit, for example, a make-up, on the keratinous materials.

Yet another aspect of the disclosure is the use of a mascara comprising a composition as defined above, in order to obtain a lengthening in the continuation of the eyelashes and/or in order to mimic the continuation of the eyelashes.

Still another aspect of the invention is the use of substantially rectilinear rigid fibres in a composition comprising, in a physiologically acceptable medium, at least one compound chosen from film-forming polymers and waxes, the composition having a thixotropic plastic behaviour, in order to obtain a lengthening in the continuation of the eyelashes and/or in order to mimic the continuation of the eyelashes.

In accordance with the disclosure, the composition comprises substantially rectilinear rigid fibres.

It will be understood that, where physicochemical values are given in the present disclosure, including the claims, the values are, unless otherwise specified, measured at ambient temperature (25° C.).

The term “fibre” should be understood to mean an object having a length L and a diameter D such that L is considerably greater than D, D being the diameter of the circle in which the section of the fibre is inscribed. For example, the ratio L/D (or aspect ratio) is chosen from the range of from 3.5 to 2 500, such as from 5 to 500, and for instance from 5 to 150.

For example, at least 50% in numerical terms, such as 75% in numerical terms, and for instance, at least 90% in numerical terms of the fibres may be such that the angle formed between the tangent to the longitudinal central axis of the fibre at an end of the fibre and the line joining the said end to the point on the longitudinal central axis of the fibre corresponding to half the length of the fibre is less than 15° and the angle formed between the tangent to the longitudinal central axis of the fibre at a point situated halfway along the fibre and the line joining one of the ends to the point on the longitudinal central axis of the fibre corresponding to half the length of the fibre is less than or equal to 15° for an identical length of fibre ranging from 0.8 mm to 5 mm, for example ranging from 1 mm to 4 mm, such as ranging from 1 mm to 3 mm, and for further example, 2 mm.

For instance, the abovementioned angle is measured at the two ends of the fibre and at a point situated halfway along the fibre, in other words, three measurements are made in this case and the mean of the angles measured is less than or equal to 15°.

For example, the tangent, at any point of the fibre, forms an angle of less than 15°.

In the present disclosure, the angle formed by the tangent at a point of the fibre is the angle formed between the tangent to the longitudinal central axis of the fibre at the said point of the fibre and the line joining the end of the fibre which is closest to the said point to the point on the longitudinal central axis of the fibre corresponding to half the length of the fibre.

Generally, the fibres incorporated into the composition have the same length of fibre or a substantially identical length.

According to the disclosure, for example, when observing under a microscope, with a lens allowing a magnification of 2.5 and with a full field of vision, a medium in which the fibres are dispersed at a fibre concentration of 1% by weight, a predominant number of fibres, that is to say at least 50% of the fibres in numerical terms, such as at least 75% of the fibres in numerical terms, and for instance at least 90% of the fibres in numerical terms, should satisfy the angular condition defined above. The measurement leading to the value of the angle is made for an identical length of fibre, this length ranging from 0.8 mm to 5 mm, for instance from 1 mm to 4 mm, such as from 1 mm to 3 mm, and for example 2 mm.

The medium in which the observation may be made is a dispersing medium allowing good dispersion of the fibres, for example water, an aqueous gel of clay and/or of associative polyurethane. It is even possible to make a direct examination of the composition comprising the fibres. A sample of the composition or of the dispersion prepared is placed between a slide and a glass coverslip for microscope examination with a lens allowing a magnification of 2.5 and with a full field of vision. The full field of vision makes it possible to see the fibres in their entirety.

The fibres incorporated into the compositions of the disclosure can also be defined as being rigid fibres, unlike the fibres of the prior art compositions, which are not rigid fibres and form, as a result, fairly large loops of curvature upon microscope examination.

The fibres of the compositions of the disclosure, which are initially substantially straight, when they are placed in a dispersing medium, do not have their shape substantially modified, which results in the angular condition defined above, reflecting a shape which can be described as substantially straight and linear. This angle condition reflects the rigidity of the fibres which can hardly be expressed by another parameter than that chosen according to the disclosure for objects having a size as small as the fibres used in the compositions of the disclosure.

For example, the angle condition that the fibres of the composition of the disclosure must satisfy illustrates the retention of the shape of the fibres, which remains substantially rectilinear, because of the rigidity of the fibre.

With the prior art fibres, which do not exist in the form of “straight sticks” and which do not therefore satisfy the angle condition for the fibres of the compositions of the disclosure, the effect of lengthening of the eyelashes cannot be obtained and is not therefore optimized.

The prior art fibres which are flexible may not have the initially rectilinear shape and when they are placed in a dispersing medium such as a cosmetic composition, these fibres may become deformed while forming loops, and offer no lengthening effect, and give an inaesthetic appearance to the eyelash. For the purposes of comparison, the fibres according to the disclosure may be compared to spaghetti before cooking, which is rigid, rectilinear and retains its shape, while the flexible, prior art fibres could be compared to cooked spaghetti which becomes deformed, bent, and cannot maintain a rectilinear shape.

The compositions of the disclosure, incorporating the specific fibres described above, possess excellent cosmetic properties, such as they allow a homogeneous and precise make-up, in the case of mascaras. For example, when the composition is applied to the eyelashes, the rigid fibres are not randomly arranged, but are placed in the continuation of the eyelashes. The composition according to the disclosure confers a very good effect of lengthening on the eyelashes, by virtue of the particular fibres which it contains and which become placed in the continuation of the eyelash. The composition of the disclosure also makes it possible to make a true “prosthesis” for the eyelash, which is may not be done with the prior art compositions that contain non-rigid fibres.

The mascara compositions according to the disclosure allow a lengthening and an optimum aesthetic effect, even in the case of bushy and/or long and/or curly eyelashes, for which particularly inaesthetic effects would be obtained with the prior art compositions comprising non-rigid, curly fibres.

The mascara compositions according to the disclosure produce a continuity of the eyelash, such that the fibres can no longer be seen with the naked eye because they are in the exact continuation of the eyelash and “mimic” it.

These fibres can be unitary or organized, for example plaited. Their shape or morphology can be of any type, for instance of a circular or polygonal (square, hexagonal or octagonal) section according to the specific application envisaged. For example, the cross section of the fibre (section perpendicular to the axis of the direction of the length of the fibre) does not have a longer length L1 and a shorter length L2 (L2 corresponds to the thickness of the fibre) such that L1/L2 (the ratio L1/L2 is also called flattening factor) is greater than or equal to 4.

For instance, their ends may be blunt and/or smooth in order to avoid injury.

For example, the fibres may have a length (L) ranging from 0.8 mm to 5 mm, such as ranging from 1 mm to 4 mm and for instance from 1 mm to 3 mm. Their section may be contained in a circle having a diameter (D) ranging from 2 nm to 500 μm, such as ranging from 100 nm to 100 μm and for instance from 1 μm to 50 μm.

The linear density of the fibres is often given in denier. The denier is the weight in grams per 9 km of thread. For example, the fibres used according to the disclosure may have a linear density ranging from 0.15 to 30 denier, and for instance from 0.18 to 18 denier.

In one aspect of the disclosure, the fibres are insoluble in water at 25° C.

The rigid fibres may be chosen from the fibres of a synthetic polymer chosen from polyesters, polyurethanes, acrylic polymers, polyolefins, polyamides, such as nonaromatic polyamides and aromatic polyimides-amides. For instance, the rigid fibres are not fibres comprising several alternate layers of polymers having different refractive indices.

As non-limiting examples of rigid fibres, there may be mentioned the fibres:

of polyesters, such as those obtained by cutting of threads sold under the names FIBRE 255-100-R11-242T SIZE 3 mm (octalobed section), FIBRE 265-34-R11-56T SIZE 3 mm (round section), FIBRE COOLMAX 50-34-591 SIZE 3 mm (tetralobed section) by the company DUPONT DE NEMOURS;

of polyamide, such as those sold under the names TRILOBAL NYLON 0.120-1.8 DPF; TRILOBAL NYLON 0.120-18 DPF; NYLON 0.120-6 DPF by the company Cellusuede products; or obtained by cutting threads sold under the name FIBRE NOMEX BRAND 430 SIZE 3 MM by the company DUPONT DE NEMOURS;

of polyimide-amide, such as those sold under the name “KERMEL”, “KERMEL TECH” by the company RHODIA;

of poly(p-phenylene-terephthalamide) (or aramide) for example, sold under the name Kevlar® by the company DUPONT DE NEMOURS;

of fibres having a multilayer structure comprising alternate layers of polymers chosen from polyesters, acrylic polymers, polyamides, such as those described in the documents EP-A-6921217, EP-A-686858 and U.S. Pat. No. 5,472,798. Such fibres are sold under the names “Morphotex”, “Teijin Tetron Morphotex” by the company TEIJIN.

For further example, the rigid fibres which may be used are the aromatic polyimide-amide fibres.

The aromatic polyimides-amides that may be used in the composition of the fibres according to the disclosure may be any aromatic polyimide-amide, but generally comprise a repeating unit corresponding to the following general formula (I):

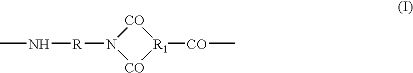

These aromatic polyimides-amides may optionally additionally comprise a repeating unit (“amide” unit) of formula (II):

—NH—R—NH—CO—R₂—CO— (II)

These aromatic polyimides-amides may optionally additionally comprise a repeating unit (“amide” unit) of formula (III):

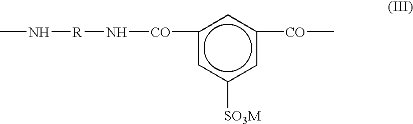

These aromatic polyimides-amides may optionally additionally comprise a repeating unit (“amide” unit) of formula (IV):

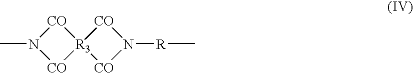

wherein R may be chosen from a divalent aromatic groups, R₂ may be chosen from divalent aromatic groups, R₃ is chosen from tetravalent aromatic groups, R₁ is chosen from trivalent aromatic groups, and M is chosen from alkali and alkaline-earth metals.

For example, R and R₂, which may be identical or different, are chosen from divalent groups comprising at least one optionally substituted aromatic ring having from 6 to 10 carbon atoms and/or an optionally substituted heterocycle with an aromatic character having from 5 to 10 atoms and comprising at least one heteroatom chosen from S, N and O; R₁ may be chosen from trivalent groups comprising at least one optionally substituted aromatic carbon ring having from 6 to 10 carbon atoms and/or an optionally substituted heterocycle with an aromatic character having from 5 to 10 atoms and comprising at least one heteroatom chosen from S, N and O.

In formula (I) cited above, R₁ may be for example, chosen from benzene rings optionally substituted with one or two substituents chosen from alkyl and alkoxy groups ranging from 1 to 10 carbon atoms, halogen atoms, nitro groups, and sulphonyl groups. Or, R₁ may be chosen from several benzene rings optionally substituted with at least one substituent chosen from alkyl and alkoxy groups ranging from 1 to 10 carbon atoms, halogen atoms, nitro groups and sulphonyl groups; for example R₁ may comprise from 2 to 5 rings, linked to each other by a single bond or by a divalent group, it being possible for the linkage of the rings to be independently in meta or in para.

Divalent groups linking the rings may be chosen for example from:

divalent groups derived from linear and/or branched alkyl groups (for example, alkylidene or alkylene groups) ranging from 1 to 10 carbon atoms optionally substituted, for instance, on the same carbon, with at least one halogen chosen from F, Cl, Br and I, and/or with at least one hydroxyl group. For example, the divalent groups may be a divalent group derived from perfluorinated alkyl groups, for example perfluorinated alkylene;

heteroatoms chosen from O, S;

divalent groups chosen from:

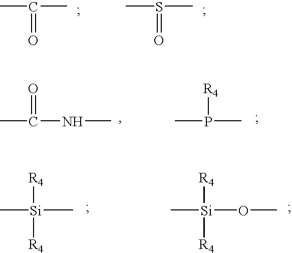

wherein R₄ is chosen from alkyl groups containing from 1 to 10 carbon atoms such as methyl, ethyl, isopropyl, and the like.

R₁ may also be chosen from condensed polycyclic carbon groups optionally substituted with at least one substituents chosen from alkyl groups and alkoxy groups containing from 1 to 10 carbon atoms, halogen atoms, nitro groups and sulphonyl groups, wherein the polycyclic carbon groups may comprise for example, from 2 to 5 benzene rings chosen for example from naphthalene, phenanthrene, coronene, perylene, phenylindane, and the like.

R₁ may also be chosen from heterocycles and condensed heterocycles with an aromatic character such as thiophene, pyrazine, pyridine, furan, quinoline, quinoxaline, isobenzofuran, wherein the heterocycles may be optionally substituted with at least one substituent chosen from alkyl groups (for example methyl, ethyl, isopropyl, and the like) and alkoxy groups ranging from 1 to 10 carbon atoms, halogen atoms (such as F, Cl, Br, I), nitro groups and sulphonyl groups.

Among the polyimides-amides that may be used in the context of the invention, non-limiting mention may be made of those in which R₁ is chosen from a benzene ring, a set of two benzene rings linked to each other by an oxygen bridge, and a naphthalene ring.

Further suitable non-limiting examples of R₁ are:

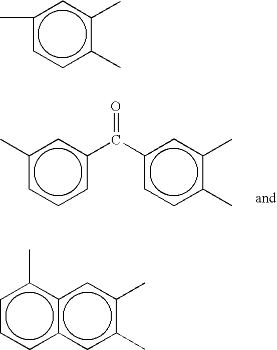

The group R₃ corresponds to the same definition as the group R₁, the only difference being that R₃ is a tetravalent and not trivalent group.

R and R₂, which may be identical or different, each may be chosen from, for example, divalent benzene rings with a meta or para linkage, optionally substituted with at least one substituent chosen from alkyl and alkoxy groups containing from 1 to 10 carbon atoms such as methyl, ethyl, isopropyl, butyl, methoxy, and the like, halogen atoms, nitro groups, and sulphonyl groups; and several benzene rings optionally substituted with at least one substituent chosen from alkyl and alkoxy groups ranging from 1 to 10 carbon atoms, halogen atoms, nitro groups, and sulphonyl groups, for example R and R₂ may comprise from 2 to 5 rings, linked to each other by a single bond or by a divalent group.

The divalent groups linking the benzene rings of R or R₂ may be chosen for example from:

divalent groups derived from linear and/or branched alkyl groups (for example alkylidene or alkylene groups) ranging from 1 to 10 carbon atoms optionally substituted, for instance on the same carbon, with at least one halogen chosen from F, Cl, Br and I and/or with at least one hydroxyl group, for example the divalent group may be a divalent group derived from perfluorinated alkyl groups, for example perfluorinated alkylene;

heteroatoms chosen from O, S;

divalent groups chosen from:

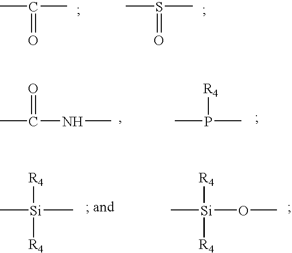

wherein R4 is chosen from alkyl groups containing from 1 to 10 carbon atoms such as methyl, ethyl, isopropyl, and the like.

R and R₂ may also each be chosen from divalent condensed polycyclic carbon groups optionally substituted with at least one substituent chosen from alkyl groups and alkoxy groups containing from 1 to 10 carbon atoms, halogen atoms, nitro groups and sulphonyl groups, wherein the polycyclic carbon groups may comprise, for example, from 2 to 5 benzene rings and may be chosen, for example, from naphthalene, phenanthrene, coronene, perylene, phenylindane, and the like.

R₂ may also be chosen from heterocycles and condensed heterocycles with an aromatic character such as thiophene, pyrazine, pyridine, furan, quinoline, quinoxaline, and isobenzofuran, wherein the heterocycles may be optionally substituted with at least one substituent chosen from alkyl groups and alkoxy groups containing from 1 to 10 carbon atoms, for example methyl, ethyl, isopropyl, and methoxy, halogen atoms (such as F, Cl, Br, I), nitro groups, and sulphonyl groups.

For example, mention may be made of polyimides-amides in which R is chosen from diphenylmethane groups and R₂ is a phenyl-1,4-diyl group; or R is chosen from diphenyl ether groups, and R₂ is a phenyl-1,4-diyl group.

Further examples of the group R₁ which have already been given above include:

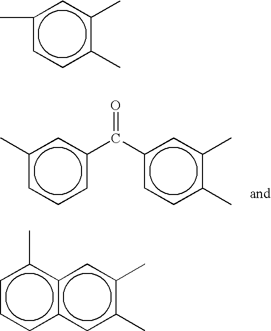

Further examples of the group R₃ include:

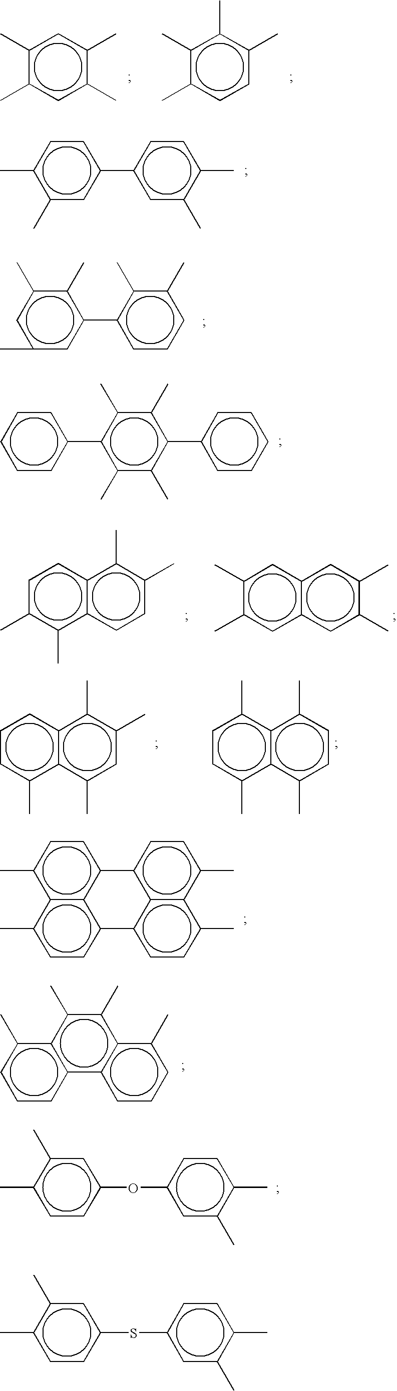

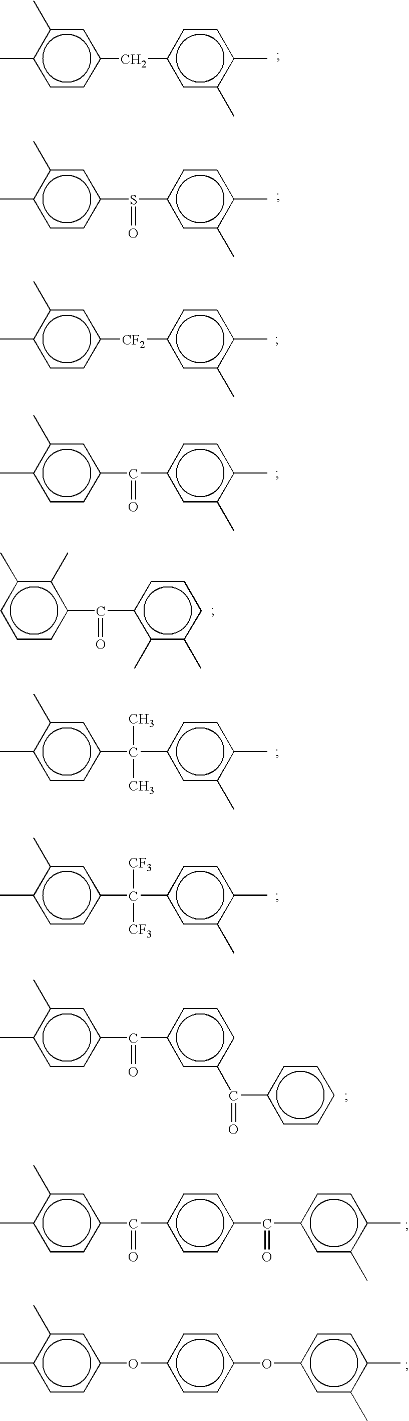

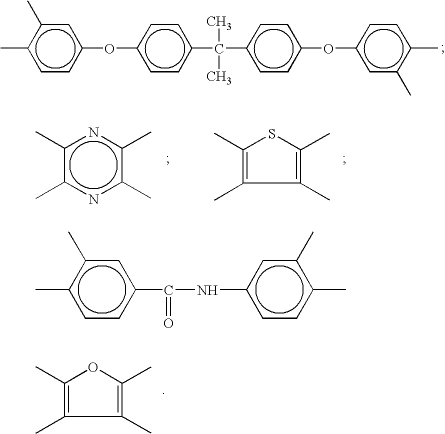

It should be noted that other examples of R₁ groups are the trivalent equivalents of the tetravalent groups R₃ exemplified above.

Additional examples of the groups R and R₂ include the following:

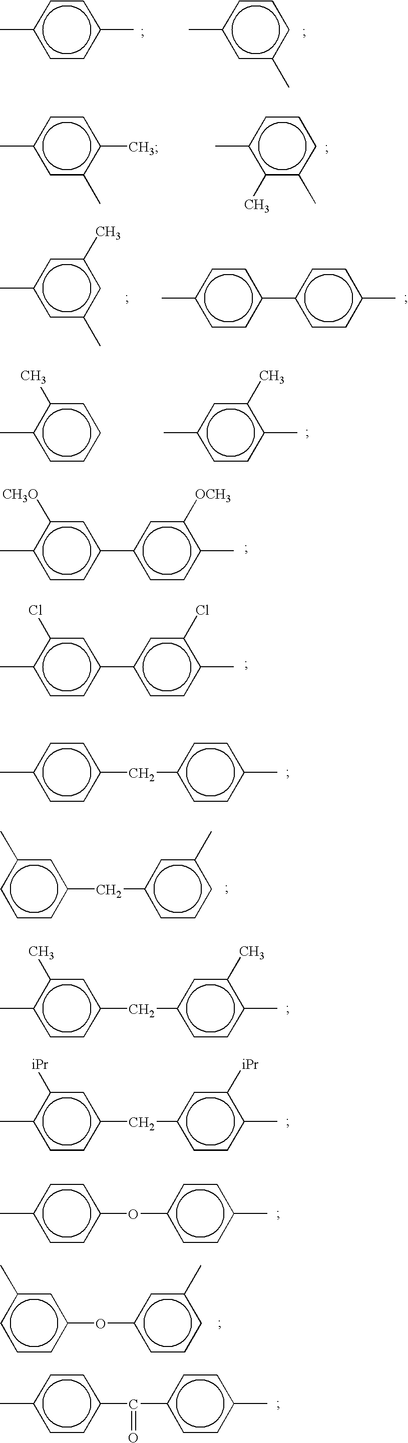

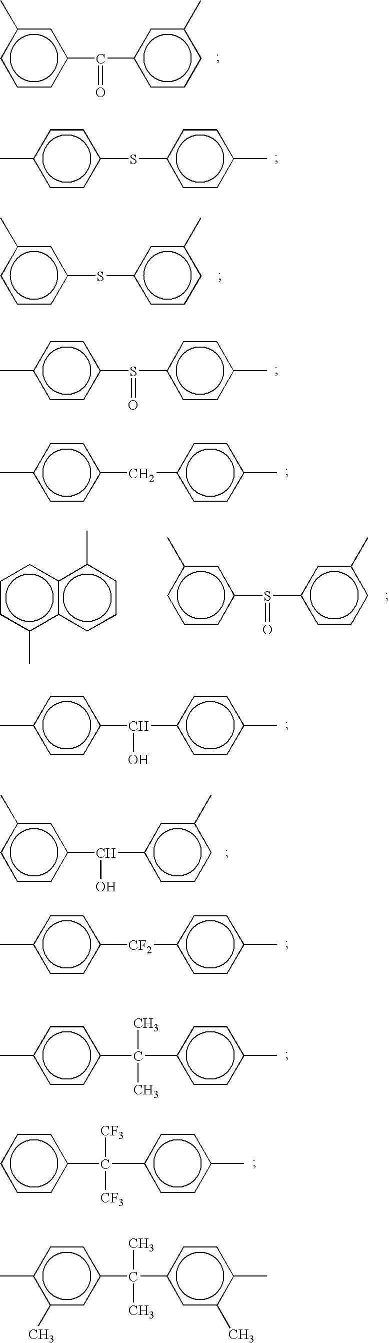

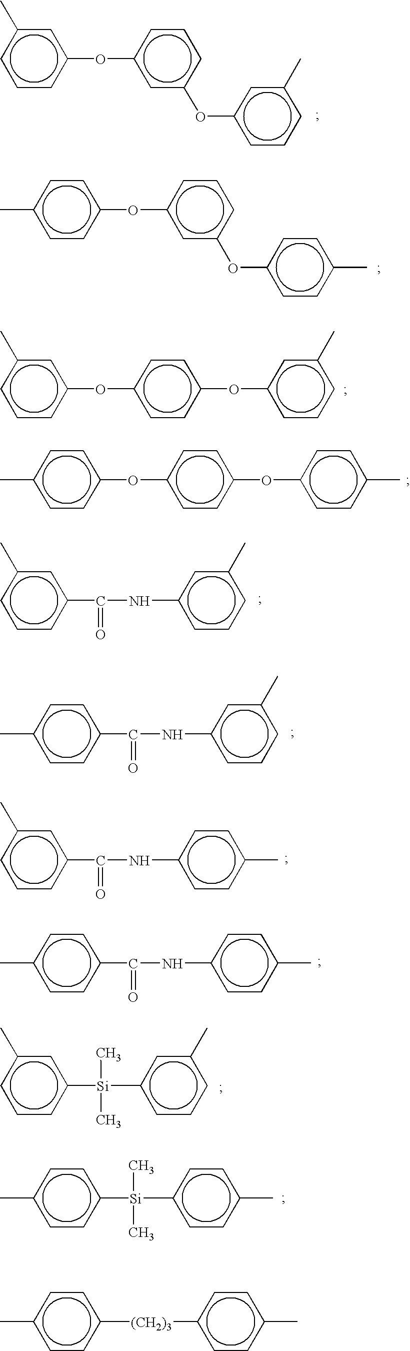

The aromatic polyimides-amides forming the fibres used in the composition of the disclosure may be obtained by any methods known to persons skilled in the art for preparing aromatic polyimides-amides, for example, by reacting a diisocyanate with trimellitic anhydride.

Polyimide-amide threads or fibres, which may be used for the compositions of the disclosure, are described for example in the document by R. PIGEON and P. ALLARD, Chimie Macromoléculaire Appliquée, 40/41 (1974), pages 139-158 (No. 600), or alternatively in the documents U.S. Pat. No. 3,802,841, FR-A-2 079 785, EP-A1-0 360 728, EP-A-0 549 494, to which reference may be made.

In order to be incorporated into the composition of the disclosure, the filaments are then cut into fibres of the desired length mentioned above.

As mentioned above, an example of aromatic polyimide-amide fibres that may be used are KERMEL TECH® fibres, in which the polyimide-amide comprises repeating units of formula:

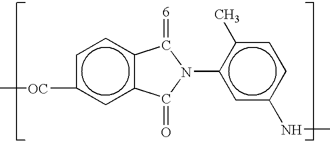

and can be obtained by polycondensation of tolylene diisocyanate and trimellitic anhydride.

The rigid fibres may be present in the composition according to the disclosure in an amount ranging from 0.01% to 10% by weight, relative to the total weight of the composition, for example, from 0.1% to 5% by weight, such as from 0.3% to 3% by weight.

The composition according to the disclosure has a thixotropic plastic behaviour.

The expression “composition having a thixotropic plastic behaviour” is understood to mean, in the present disclosure, a composition having the following properties:

the composition has a rheofluidizing character, that is to say that the viscosity of the composition decreases when increasing shearings are applied to the composition;

the composition, after the application of an intense shearing, fluidizes (for example, its viscosity decreases) but the destructuration of the composition is delayed in time. For instance, the viscosity, the consistency, and the elasticity of the composition after its destructuration, such as after a rest time of one minute after having applied the shearing, are less than those of the composition before the application of the intense shearing;

the composition regenerates its initial structure partly or completely only after a sufficient rest time. The restructuring of the composition therefore does not occur instantly but in a deferred manner over time. For instance, the composition, when subjected to a constant shearing of 1 000 s⁻¹ for one minute, partly or completely returns to its initial viscosity after a sufficient rest time which may be longer or shorter.

A definition of a thixotropic composition is, for example, indicated in the collection “Comprendre la Rhéologie—De la circulation du sang à la prise du béton” by P. Cousot and J. L. Grossiord, EDP Science, 2002, pages 16 and 17.

The thixotropic plastic behaviour of the composition may be evaluated according to the protocol for evaluation of thixotropic character described below.

The thixotropic plastic behaviour of the composition may be characterized by the consistency (G*), the elasticity (δ) and the yield point (τ_{C}); these parameters are defined, for example, in the collection “Initiation à la Rhéologie”, G. Couarraze and J. L. Grossiord, 2nd edition, 1991, Edition Lavoisier-Tec 1 Doc.

These parameters can be determined by measurements carried out at 25° C.±0.5° C. with the aid of the applied stress Haake rheometer 75 from the company Thermo Rheo, equipped with a stainless steel rotor having a flat/flat geometry, the plane having a diameter of 20 mm and a clearance gap (distance between the lower plane—called stator plane—on which the composition is deposited, and the upper plane—called rotor plane) of 0.3 mm. The two planes are grooved in order to limit the phenomena of slipping on the walls of the planes.

a) First measured, in an oscillatory mode, are the Theological characteristics of the composition under a low shearing that avoids the destructuration of the composition (conditions considered as making it possible to evaluate the rheological characteristics of the composition at rest) by applying to the composition a harmonic shearing according to a stress τ(t) varying sinusoidally according to a pulsation ω(ω=2Π N, N being the frequency of the shearing applied). The composition thus sheared is subjected to a stress τ(t) and responds according to a deformation γ(t) corresponding to microdeformations for which the consistency varies little as a function of the applied stress.

The stress τ(t) and the deformation γ(t) are defined respectively by the following relationships:

τ(t)=τ₀ cos ωtγ(t)=γ₀ cos(ωt−δ)

τ₀ being the maximum amplitude of the stress and γ₀ being the maximum amplitude of the deformation.

The measurements are carried out at a frequency of 1 Hz (N=1 Hz).

The variation of the consistency G* (corresponding to the ratio of the applied stress to the measured deformation) and the elasticity δ (corresponding to the angle of phase displacement of the applied stress relative to the measured deformation) is thus measured as a function of the applied stress τ(t).

The deformation of the composition is measured, for example, for the zone of stress for which the initial consistency G*ᵢ and the initial elasticity δᵢ vary little (zone of microdeformations in which the variation of the initial consistency and of the initial elasticity is less than 15%) and the initial consistency G*ᵢ is thus determined.

b) Next, the composition is destructured by applying an intense continuous shearing \dot{γ} of 1 000 s⁻¹ for 60 seconds.

c) After the destructuring of the composition, the restructuring of the composition is monitored as a function of the rest time by applying to the composition a harmonic shearing in an oscillatory mode according to a very weak stress varying sinusoidally at a frequency of 1 Hz, the stress being such that the deformation of the composition corresponds to microdeformations for which the consistency of the composition varies little (the variation in the consistency is less than 15%) as a function of this applied stress.

The consistency G* of the composition is then measured as a function of the rest time; the value of the consistency is then determined after a rest time of one minute (G₁*) and after a rest time of 30 minutes (G₃₀*).

The resumption of thixotropy of the composition is then determined after x minutes corresponding to the ratio: 100×(G*ᵢ−Gₓ*)/Gₓ*.

The resumption of thixotropy after 1 minute of rest is calculated with the value G₁*(x=1); the resumption of thixotropy after 30 minutes is calculated with the value G₃₀*(x=30).

The thixotropic plastic behaviour of the composition is, for instance, characterized by an initial consistency Gᵢ* ranging from 1×10² Pa to 1×10⁵ Pa, such as ranging from 5×10² Pa to 5×10⁴ Pa, and for example ranging from 6×10² Pa to 9×10³ Pa measured under a sinusoidal stress at a frequency of 1 Hz.

In addition, the composition may have an initial elasticity δᵢ that may range from 1° to 45°, such as ranging from 10° to 35°.

For example, the composition has a yield point τ_{C} ranging from 10 Pa to 3 500 Pa, and such as ranging from 20 Pa to 1 000 Pa, which means that the composition according to the disclosure does not run under its own weight but that it is necessary to apply to the composition a critical stress above which its flow is brought about.

For instance, the composition, after having been subjected to a continuous shearing of 1 000 s⁻¹ for one minute, has a resumption of thixotropy:

(i) of less than or equal to 20%, such as ranging from 0.1% to 20%, after 1 minute of rest

(ii) of less than or equal to 90%, such as ranging from 20% to 90%, after 30 minutes of rest.

The composition according to the disclosure may comprise an aqueous medium, constituting an aqueous phase, which may be the continuous phase of the composition.

The composition may comprise water and optionally a hydrophilic organic solvent (a water-miscible organic solvent) such as alcohols and for instance monoalcohols having from 2 to 5 carbon atoms such as ethanol, isopropanol or n-propanol, polyols having from 2 to 8 carbon atoms such as glycerin, diglycerin, propylene glycol, ethylene glycol, 1,3-butylene glycol, sorbitol, penthylene glycol, C₃-C₄ ketones, C₂-C₄ aldehydes. The water or the mixture of water and hydrophilic organic solvent(s) may be present in the composition according to the disclosure in an amount ranging from 0.1% to 90% by weight, relative to the total weight of the composition, such as from 0.1% to 60% by weight.

The composition may also comprise an oily medium, or a liquid fatty phase, comprising a fatty substance chosen from oils, organic solvents, and mixtures thereof. The fatty phase may form a continuous phase of the composition. For example, the composition according to the disclosure may be anhydrous.

The liquid fatty phase may be comprised, for example, of any physiologically acceptable, and for instance cosmetically acceptable, oil chosen from oils of mineral, animal, plant or synthetic origin, carbonaceous, hydrocarbonaceous, fluorinated and/or silicone oils, alone or as a mixture.

The total liquid fatty phase of the composition may be present in an amount ranging from 0.1% to 98% by weight, relative to the total weight of the composition, such as from 1 to 80% by weight.

For example, the liquid fatty phase of the composition may comprise at least one volatile oil or organic solvent and/or at least one nonvolatile oil.

The expression “volatile oil or organic solvent” is understood to mean, for the purposes of the disclosure, any nonaqueous medium capable of evaporating in contact with skin or keratinous fibres in less than one hour, at room temperature and atmospheric pressure. The volatile organic solvent(s) and the volatile oils of the disclosure are organic solvents and volatile cosmetic oils, which are liquid at room temperature, having a nonzero vapour pressure, at room temperature and atmospheric pressure, ranging from 0.13 Pa to 40 000 Pa (10⁻³ to 300 mmHg), such as ranging from 1.3 Pa to 13 000 Pa (0.01 to 100 mmHg), and for instance, ranging from 1.3 Pa to 1 300 Pa (0.01 to 10 mmHg). The expression “nonvolatile oil” is understood to mean an oil which remains on skin or keratinous fibres at room temperature and atmospheric pressure for at least several hours and which has for example, a vapour pressure of less than 10⁻³ mmHg (0.13 Pa).

These oils may be chosen from, for example, hydrocarbon oils, silicone oils, fluorinated oils, and mixtures thereof.

The expression “hydrocarbon oil” is understood to mean an oil comprising mainly hydrogen and carbon atoms and optionally oxygen, nitrogen, sulphur and phosphorus atoms. The volatile hydrocarbon oils may be chosen from hydrocarbon oils having from 8 to 16 carbon atoms and, for example, branched C₈-C₁₆ alkanes, such as C₈-C₁₆ isoalkanes of petroleum origin (also called isoparaffins) such as isododecane (also called 2,2,4,4,6-pentamethylheptane), isodecane, isohexadecane, and for example the oils sold under the trade names Isopars' or Permetyls, C₈-C₁₆ branched esters, isohexyl neopentanoates, and mixtures thereof. Other volatile hydrocarbon oils such as petroleum distillates, for instance those sold under the name Shell Solt by the company SHELL, may also be used. For example, the volatile solvent may be chosen from volatile hydrocarbon oils having from 8 to 16 carbon atoms and mixtures thereof.

As volatile oils, there may also be used volatile silicones, such as for example volatile linear or cyclic silicone oils, for example those having a viscosity≦6 centistokes (6×10⁻⁶ m²/s), and having, for instance, from 2 to 10 silicon atoms, these silicones optionally comprising alkyl or alkoxy groups having from 1 to 22 carbon atoms. As volatile silicone oils which can be used in accordance with the present disclosure, there may be mentioned for example, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

Fluorinated volatile solvents such as nonafluoromethoxybutane or perfluoromethylcyclopentane may also be used.

The volatile oil may be present in the composition according to the disclosure in an amount ranging from 0.1% to 98% by weight, relative to the total weight of the composition, such as from 1% to 65% by weight.

The composition may also comprise at least one nonvolatile oil such as those chosen from nonvolatile hydrocarbon, silicone, and fluorinated oils.

As non-limiting examples of nonvolatile hydrocarbon oils, there may be mentioned:

hydrocarbon oils of plant origin such as triglycerides comprising esters of fatty acids and of glycerol in which the fatty acids may have varying chain lengths ranging from C₄ to C₂₄, it being possible for said chains to be linear or branched, saturated or unsaturated; these oils can be those such as wheatgerm oil, sunflower oil, grapeseed oil, sesame oil, maize oil, apricot oil, castor oil, karite oil, avocado oil, olive oil, soyabean oil, sweet almond oil, palm oil, rapeseed oil, cottonseed oil, hazelnut oil, macadamia oil, jojoba oil, lucerne oil, poppyseed oil, pumpkinseed oil, sesame oil, gourd oil, rapeseed oil, blackcurrant seed oil, evening primrose oil, millet oil, barley oil, quinoa oil, rye oil, safflower oil, candlenut oil, passionflower oil, rose-muscat oil; or alternatively triglycerides of caprylic/capric acids such as those sold by the company Stéarineries Dubois or those sold under the names Miglyol 810, 812 and 818 by the company Dynamit Nobel,

synthetic ethers having from 10 to 40 carbon atoms;

linear or branched hydrocarbons of mineral or synthetic origin such as petroleum jelly, polydecenes, hydrogenated polyisobutene such as parleam, squalane, and mixtures thereof;

synthetic esters such as the oils of formula R₁COOR₂ in which R₁ may be chosen from the residues of a linear or branched fatty acid comprising from 1 to 40 carbon atoms and R₂ may be chosen from hydrocarbon chains, such as branched hydrocarbon chains, comprising from 1 to 40 carbon atoms provided that R₅+R₆ is ≧10, such as for example Purcellin oil (ketostearyl octanoate), isopropyl myristate, isopropyl palmitate, C₁₂ to C₁₅ alcohol benzoate, hexyl laurate, diisopropyl adipate, isononyl isononanoate, 2-ethylhexyl palmitate, isostearate isostearate, octanoates, decanoates or ricinoleates of alcohols or of polyalcohols such as propylene glycol dioctanoate; hydroxylated esters such as isostearyl lactate, diisostearyl malate; and esters of pentaerythritol;

fatty alcohols which are liquid at room temperature comprising branched and/or unsaturated carbon chains having from 12 to 26 carbon atoms such as octyl dodecanol, isostearyl alcohol, oleyl alcohol, 2-hexyldecanol, 2-butyloctanol, 2-undecylpentadecanol;

higher fatty acids such as oleic acid, linoleic acid, linolenic acid; and mixtures thereof.

The nonvolatile silicone oils which may be used in the composition according to the disclosure include nonvolatile polydimethylsiloxanes (PDMS), polydimethylsiloxanes comprising alkyl or alkoxy groups, pendent and/or at the silicone chain end, groups each having from 2 to 24 carbon atoms, phenylated silicones such as phenyltrimethicones, phenyldimethicones, phenyltrimethylsiloxydiphenylsiloxanes, diphenyldimethicones, diphenylmethyldiphenyltrisiloxanes, (2-phenylethyl)trimethylsiloxy-silicates.

The fluorinated oils which may be used in accordance with the present disclosure include, for example, fluorosilicone oils, fluorinated polyethers, and fluorinated silicones as described in EP-A-847752.

The nonvolatile oil may be present in the composition according to the disclosure in an amount ranging from 0.1% to 80% by weight, such as from 0.1% to 50% by weight, relative to the total weight of the composition, and for instance from 0.1% to 20% by weight.

The composition according to the disclosure may comprise a thixotropic thickening agent in a sufficient quantity to confer the thixotropic plastic behaviour on the composition.

The thixotropic thickening agent may be present in an amount ranging from 0.5% to 15% by weight, relative to the total weight of the composition, for example ranging from 1% to 15% by weight, such as ranging from 2% to 10% by weight, and for further example ranging from 2% to 8% by weight.

The thickening agent is chosen according to the medium for the composition: if the composition comprises an aqueous medium, a thickener for an aqueous medium is then used. If the composition comprises an oily medium, a thickener for an oily medium is then used. When the composition is in the form of an emulsion, the thixotropic thickening agent may be present in, for example, the external phase of the emulsion.

The thickening agent for the aqueous medium may be chosen from hydrophilic clays, carrageenan gum, and hydrophilic pyrogenic silica.

The expression hydrophilic clay is understood to mean a clay capable of swelling in water, such as a clay that swells in water and forms a colloidal dispersion after hydration.

Clays are products well known per se, and are described for example in the work “Minéralogie des Argiles, S. Caillère, S. Hénin, M. Rautureau, 2nd edition 1982, Masson”, the teaching of which is included herein by way of reference.

Clays are silicates comprising a cation which may be chosen from calcium, magnesium, aluminium, sodium, potassium and lithium cations, and mixtures thereof.

By way of examples of such products, there may be mentioned clays of the family of smectites such as montmorillonites, hectorites, bentonites, beidellites and saponites, and of the family of vermiculites, stevensite, chlorites.

These clays may be of natural or synthetic origin. For example, clays may be used which are cosmetically compatible and acceptable with keratinous materials such as the eyelashes and the skin.

As hydrophilic clays, there may be mentioned smectites such as saponites, hectorites, montmorillonites, bentonites, and beidellite.

As hydrophilic clays, there may be mentioned synthetic hectorites (also called laponites) such as the products sold by the company Laporte under the name Laponite XLG, Laponite RD, Laponite RDS (these products are sodium and magnesium silicates such as sodium, lithium and magnesium silicates); bentonites such as the product sold under the name Bentone HC by the company RHEOX; magnesium and aluminium silicates, for instance those that are hydrated, such as the products sold by the company Vanderbilt Company under the name Veegum ultra, Veegum HS, Veegum DGT, or alternatively calcium silicates such as that in synthetic form sold by the company under the name Microcel C.

The hydrophilic pyrogenic silicas may be obtained by high temperature hydrolysis of a volatile compound of silicon in an oxyhydrogen flame, producing a finely divided silica. Hydrophilic silicas have a large number of silanol groups at their surface. Such hydrophilic silicas are for example marketed under the names “AEROSIL 130®”, “AEROSIL 200®”, “AEROSIL 255®”, “AEROSIL 300®”, “AEROSIL 380®” by the company Degussa, “CAB-O-SIL HS-5®”, “CAB-O-SIL EH-5®”, “CAB-O-SIL LM-130®”, “CAB-O-SIL MS-55®”, and “CAB-O-SIL M-5®” by the company Cabot.

For example, the hydrophilic pyrogenic silica may have a particle size, which may be nanometric to micrometric, for example ranging from about 5 to 200 nm.

The thixotropic thickening agent for the oily medium may be chosen from organophilic clays, hydrophobic pyrogenic silicas, and elastomeric organopolysiloxanes.

The organophilic clays are clays modified by chemical compounds making the clay capable of swelling in oily media.

The clay may be chosen from montmorillonite, bentonite, hectorite, attapulgite, sepiolite, and mixtures thereof. Mention may be made of the clays chosen from bentonites and hectorites.

These clays may be modified with a chemical compound chosen from quaternary amines, tertiary amines, amine acetates, imidazolines, amine soaps, fatty sulphates, alkyl aryl sulphonates, amine oxides, and mixtures thereof.

As non-limiting examples of organophilic clays, there may be mentioned quaternium 18 bentonites such as those sold under the names Bentone 3, Bentone 38, Bentone 38V by the company Rhéox, Tixogel VP by the company United Catalyst, Claytone 34, Claytone 40, Claytone XL by the company Southern Clay; steralkonium bentonites such as those sold under the names Bentone 27 by the company Rheox, Tixogel LG by the company United Catalyst, Claytone A F, Claytone APA by the company Southern Clay; quaternium-18/benzalkonium bentonite such as those sold under the names Claytone H T, and Claytone P S by the company Southern Clay.

It is possible to chemically modify the surface of the silica via a chemical reaction generating a reduction in the number of silanol groups. For instance, it may be possible to substitute silanol groups by hydrophobic groups, and thus a hydrophobic silica could then be obtained.

Hydrophobic groups that may be used include:

trimethylsiloxyl groups, which can be obtained for example by treating pyrogenic silica in the presence of hexamethyldisilazane. Silicas thus treated are called “Silica silylate” according to CTFA (6th edition, 1995). They are for example marketed under the references “AEROSIL R812®” by the company Degussa, and “CAB-O-SIL TS-530®”, by the company Cabot.

dimethylsilyloxyl or polydimethylsiloxane groups, which may be, for instance, obtained by treating pyrogenic silica in the presence of polydimethylsiloxane or dimethyldichlorosilane. Silicas thus treated are called “Silica dimethyl silylate” according to CTFA (6th edition, 1995). They are for example marketed under the references “AEROSIL R972®”, “AEROSIL R974®” by the company Degussa, “CAB-O-SIL TS-610®”, “CAB-O-SIL TS-720®” by the company Cabot.

For instance, the hydrophobic pyrogenic silica may have a particle size which may be nanometric or micrometric, for example ranging from about 5 to 200 nm.

The elastomeric organopolysiloxanes are in general partially or completely crosslinked and optionally have a three-dimensional structure. Included in a fatty phase, they change, according to the amount of fatty phase used, from a product with a spongy appearance when they are used in the presence of small amounts of fatty phase, to a homogeneous gel, in the presence of higher quantities of fatty phase.

The elastomeric organopolysiloxanes combined with a fatty phase generally exist in the form of a gel comprising an elastomeric organopolysiloxane combined with a fatty phase, contained in at least one hydrocarbon oil and/or one silicone oil. They can be chosen for example, from the crosslinked polymers described in application EP-A-0295886.

According to the disclosure herein, the elastomeric organopolysiloxanes can be obtained by addition and crosslinking reaction of at least:

(a) one organopolysiloxane having at least two lower alkenyl groups per molecule;

(b) one organopolysiloxane having at least two hydrogen atoms linked to a silicon atom per molecule; and

(c) a platinum type catalyst.

The lower alkenyl groups may be, for example, vinyl, allyl and propenyl groups.

The platinum catalyst may be for example chloroplatinic acid, complexes containing chloroplatinic acid, and platinum supported by an appropriate support.

The elastomeric organopolysiloxanes combined with a fatty phase may also be chosen from those described in U.S. Pat. No. 5,266,321.

According to the disclosure herein, the elastomeric organopolysiloxanes may also be chosen, for example, from:

i)polyorganopolysiloxanes comprising R₂SiO and RSiO_{1.5} units and optionally R₃SiO_{0.5} and/or SiO₂ units wherein the radicals R, which may be identical or different, may be chosen from a hydrogen atom, alkyls such as methyl, ethyl and propyl, aryls such as phenyl and tolyl, unsaturated aliphatic groups such as vinyl, and wherein the weight ratio of the R₂SiO units to the RSiO_{1.5} units ranges from 1/1 to 30/1;

ii) polyorganopolysiloxanes that are insoluble and swellable in silicone oil, which can be obtained by adding an organohydrogenpolysiloxane (1) and an organopolysiloxane (2) having unsaturated aliphatic groups such that the quantity of hydrogen or of unsaturated aliphatic groups in respectively (1) and (2) ranges from 1 to 20 mol % when the organopolysiloxane is noncyclic and ranges from 1 to 50 mol % when the organopolysiloxane is cyclic.

The composition according to the disclosure may additionally comprise an additional thickening agent different from the thixotropic thickening agents described above.

When the composition according to the disclosure comprises an aqueous medium, it may therefore comprise an additional thickening agent for an aqueous medium. This thickening agent is not capable, on its own, of giving the composition the thixotropic plastic character (nonthixotropic thickener); it makes it possible for example to adjust the viscosity of the composition in order to obtain a homogeneous flow.

When the composition comprises an aqueous medium, the additional thickening agent may be chosen from hydrophilic thickening agents (nonthixotropic thickening agent for aqueous media).

Among the additional hydrophilic thickening agents which can be used according to the disclosure, there may be mentioned:

water-soluble cellulosic thickeners such as hydroxyethylcellulose, methylcellulose, hydroxypropylcellulose and carboxymethylcellulose. Among these, there may be mentioned for example, the gums sold under the name “Cellosize QP 4400 H” by the company Amercol,

guar gums, for example those sold under the name VIDOGUM GH 175 by the company UNIPECTINE, and JAGUAR C by the company MEYHALL,

quaternized guar gums sold under the name “Jaguar C-13-S” by the company Meyhall,

nonionic guar gums comprising C₁-C₆ hydroxyalkyl groups. There may be mentioned, by way of example, hydroxymethyl, hydroxyethyl, hydroxypropyl and hydroxybutyl groups. Such guar gums are for example sold under the trade names JAGUAR HP8, JAGUAR HP60 and JAGUAR HP120 and JAGUAR HP105 by the company MEYHALL, or under the name GALACTASOL 40H4FD2 by the company AQUALON,

xanthan, carob, scleroglucan, gellan, rhamsan and karaya gums,

alginates, maltodextrin, starch and its derivatives, hyaluronic acid and its salts,

polyglyceryl (meth)acrylate polymers sold under the names “Hispagel” or “Lubragel” by the companies Hispano Quimica or Guardian,

polyvinylpyrrolidones,

polyvinyl alcohols,

crosslinked polymers and copolymers of acrylamide, such as those sold under the names “PAS 5161” or “Bozepol C” by the company Hoechst, “Sepigel 305” by the company Seppic, or alternatively

crosslinked homopolymers of methacryloyloxyethyltrimethylammonium chloride sold under the name “Salcare SC95” by the company Allied Colloïd, and

associative polymers such as associative polyurethanes.

The hydrophilic additional thickening agent may be, for example, an associative polyurethane.

Associative polyurethanes are nonionic block copolymers comprising in the chain both hydrophilic sequences most often of a polyoxyethylenated nature and hydrophobic sequences which may be chosen from at least one of aliphatic linkages, cycloaliphatic linkages, and linkages.

For example, these polymers comprise at least two lipophilic hydrocarbon chains having from C₆ to C₃₀ carbon atoms, separated by a hydrophilic sequence, wherein the hydrocarbon chains may be pendent chains or chains at the end of a hydrophilic sequence. For further example, it is possible for one or more pendent chains to be provided. In addition, the polymer may contain a hydrocarbon chain at one end or at both ends of a hydrophilic sequence.

The polymers may be block polymers in triblock or multiblock form. The hydrophobic sequences can therefore be at each end of the chain (for example: triblock copolymer with a hydrophilic central sequence) or distributed both at the ends and in the chain (multiblock copolymer, for example). The polymers may also be graft or star-shaped polymers.

For instance, the polymers may be triblock copolymers whose hydrophilic sequence is a polyoxyethylenated chain comprising from 50 to 1,000 oxyethylated groups. In general, the associative polyurethanes contain a urethane bond between the hydrophilic sequences, hence the origin of the name.

By way of example of associative polymers that may be used in accordance with the present disclosure, mention may be made of the polymer C₁₆-EO₁₂₀-C₁₆ sold by the company HULS (under the name Serad FX1100, a molecule with a urethane functional group and a weight-average molecular weight of 1,300), EO being an oxyethylenated unit. As associative polymers, it is also possible to use Rheolate 205 containing a urea functional group, sold by the company RHEOX or alternatively Rheolate 208 or 204. These associative polyurethanes are sold in pure form.

The product DW 1206B from RHOM & HAAS containing a C₂₀ alkyl chain and a urethane bond, sold at a dry matter content of 20% in water, may also be used.

It is also possible to use solutions or dispersions of these polymers, such as in water or in an aqueous-alcoholic medium. By way of non-limiting example of such polymers, there may be mentioned Serad FX1010, Serad FX1035 and Serad 1070 sold by the company HULS, Rheolate 255, Rheolate 278 and Rheolate 244 sold by the company RHEOX. It is also possible to use the product DW 1206F and DW 1206J, and Acrysol RM 184 or Acrysol 44 from the company RHOM & HAAS, or alternatively Borchigel LW 44 from the company BORCHERS.

The polymers which may be used according to the disclosure are for example those described in the article by G. Fonnum, J. Bakke and Fk. Hansen—Colloid Polym. Sci 271, 380.389 (1993).

When the composition comprises an oily medium, the additional thickening agent may be chosen from lipophilic thickening agents (nonthixotropic thickening agent for oily media).

Among the additional lipophilic thickening agents that can be used according to the disclosure, there may be mentioned:

alkylated guar gums (with C₁-C₆ alkyl group), such as those described in EP-A-708114;

oil gelling polymers such as the triblock or star-shaped polymers resulting from the polymerization or copolymerization of at least one monomer with an ethylenic group, such as the polymers sold under the name Kraton;

polyamide resins comprising alkyl groups having from 12 to 22 carbon atoms, such as those described in U.S. Pat. No. 5,783,657.

The additional thickening agent may be present in an amount ranging from 0.1% to 5% by weight, relative to the total weight of the composition, for instance, ranging from 0.1% to 3% by weight.

The composition according to the disclosure comprises at least one compound chosen from film-forming polymers and waxes.

The film-forming polymer may be a polymer solubilized or dispersed in the form of particles in an aqueous phase of the composition or alternatively solubilized or dispersed in the form of particles in a liquid fatty phase. The composition may also comprise a mixture of these polymers.

The film-forming polymer may be present in the composition according to the disclosure in a dry matter content ranging from 0.1% to 60% by weight relative to the total weight of the composition, such as from 0.5% to 40% by weight, for instance, from 1% to 30% by weight.

In the present disclosure, the expression “film-forming polymer” is understood to mean a polymer capable of forming, on its own or in the presence of a film-forming aid, a continuous and adherent film on a support, for instance on the keratinous materials.

For example, a film-forming polymer may be used which is capable of forming a hydrophobic film, that is to say a polymer whose film has a solubility in water at 25° C. of less than 1% by weight.

Among the film-forming polymers which can be used in the composition of the present disclosure, there may be mentioned synthetic polymers of the free-radical type or of the polycondensate type, polymers of natural origin and mixtures thereof.

The expression free-radical film-forming polymer is understood to mean a polymer obtained by polymerization of monomers with for instance, ethylenic unsaturation, each monomer being capable of homopolymerizing (in contrast to polycondensates).

The film-forming polymers of the free-radical type may be for example, vinyl polymers or copolymers, such as acrylic polymers.

The vinyl film-forming polymers may result from the polymerization of ethylenically unsaturated monomers having at least one acid group and/or esters of these acid monomers and/or amides of these acid monomers.

As a monomer carrying an acid group, there may be used α,β-ethylenic unsaturated carboxylic acids such as acrylic acid, methacrylic acid, crotonic acid, maleic acid and itaconic acid. For example, (meth)acrylic acid and itaconic acid may be used.

The esters of acid monomers may be chosen from the esters of (meth)acrylic acid (also called (meth)acrylates), for example alkyl, such as C₁-C₃₀, for instance C₁-C₂₀, alkyl, (meth)acrylates, aryl, such as C₆-C₁₀ aryl, (meth)acrylates, hydroxyalkyl, for instance C₂-C₆ hydroxyalkyl, (meth)acrylates.

Among the alkyl (meth)acrylates, non-limiting mention may be made of methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates, non-limiting mention may be made of hydroxyethyl acrylate, 2-hydroxypropyl acrylate, hydroxyethyl methacrylate and 2-hydroxypropyl methacrylate.

Among the aryl (meth)acrylates, non-limiting mention may be made of benzyl acrylate and phenyl acrylate.

According to the present disclosure, the alkyl group of the esters may be either fluorinated or perfluorinated, that is to say that some or all of the hydrogen atoms of the alkyl group may be substituted with fluorine atoms.

As amides of the acid monomers, there may be mentioned for example (meth)acrylamides, such as N-alkyl(meth)acrylamides, and for instance, of a C₂-C₁₂ alkyl. Among the N-alkyl(meth)acrylamides, there may be mentioned N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or their esters and/or their amides, such as those mentioned above.

As examples of vinyl esters, there may be mentioned vinyl acetate, vinyl neodecanoate, vinyl pivalate, vinyl benzoate and vinyl t-butyl benzoate.

As styrene monomers, there may be mentioned styrene and alpha-methylstyrene.

It is possible to use any monomer known to a person of ordinary skill in the art entering into the categories of acrylic and vinyl monomers (including the monomers modified by a silicone chain).

Among the film-forming polycondensates, there may be mentioned polyurethanes, polyesters, polyester amides, polyamides, epoxy ester resins, and polyureas.

The polyurethanes may be chosen from anionic, cationic, nonionic and/or amphoteric polyurethanes, polyurethane-acrylics, polyurethane-polyvinylpyrrolidones, polyester-polyurethanes, polyether-polyurethanes, polyureas, and polyurea-polyurethanes.

The polyesters may be obtained, in a known manner, by polycondensation of dicarboxylic acids with polyols, such as diols.

The dicarboxylic acids may be aliphatic, alicyclic and/or aromatic. There may be mentioned as examples of such acids: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalenedicarboxylic acid. These dicarboxylic acid monomers may be used alone or in combination with at least two dicarboxylic acid monomers. Examples of monomers that may be used are phthalic acid, isophthalic acid and terephthalic acid.

The diols may be chosen from aliphatic, alicyclic and/or aromatic diols. For example, the diols may be chosen from: ethylene glycol, diethylene glycol, triethylene glycol, 1,3-propanediol, cyclohexanedimethanol and 4-butanediol. As other polyols, there may be used glycerol, pentaerythritol, sorbitol, and trimethylolpropane.

The polyester amides may be obtained in a manner similar to the polyesters, by polycondensation of diacids with diamines or amino alcohols. As diamines, there may be used ethylenediamine, hexamethylenediamine, meta- and/or para-phenylenediamine. As aminoalcohols, monoethanolamine may be used.

The polyester may, in addition, comprise at least one monomer carrying at least one —SO₃M group, wherein M is chosen from hydrogen atoms, ammonium ions NH₄⁺, and metal ions, such as for example Na⁺, Li⁺, K⁺, Mg²⁺, Ca²⁺, Cu²⁺, Fe²⁺ and Fe³⁺ions. There may also be used for instance, a bifunctional aromatic monomer comprising such an —SO₃M group.

The aromatic ring of the bifunctional aromatic monomer carrying, in addition, an —SO₃M group as described above may be chosen for example from benzene, naphthalene, anthracene, diphenyl, oxydiphenyl, sulphonyldiphenyl and methylenediphenyl rings. There may also be mentioned as examples of a bifunctional aromatic monomer carrying, in addition, an —SO₃M group: sulphoisophthalic acid, sulphoterephthalic acid, sulphophthalic acid, 4-sulphonaphthalene-2,7-dicarboxylic acid.

Mention may be made of the use of copolymers based on isophthalate/sulphoisophthalate, for instance, of copolymers obtained by condensation of diethylene glycol, cyclohexanedimethanol, isophthalic acid and sulphoisophthalic acid. Such polymers are sold, for example, under the trade name Eastman AQ® by the company Eastman Chemical Products.

The optionally modified polymers of natural origin may be chosen from shellac resin, sandarac gum, dammars, elemis, copals, cellulosic polymers and mixtures thereof.

According to an aspect of the composition according to the disclosure, the film-forming polymer may be present in the form of particles in aqueous dispersion, generally known as latex or pseudolatex. The techniques for preparing these dispersions are well known to persons skilled in the art.

As an aqueous dispersion of film-forming polymer, there may be used the acrylic dispersions sold under the names NEOCRYL XK-90®, NEOCRYL A-1070®, NEOCRYL A-1090®, NEOCRYL BT-62®, NEOCRYL A-1079®, NEOCRYL A-523® by the company AVECIA-NEORESINS, DOW LATEX 432® by the company DOW CHEMICAL, DAITOSOL 5000 AD® by the company DAITO KASEY KOGYO; or else the aqueous dispersions of polyurethane which are sold under the names NEOREZ R-981®, NEOREZ R-974® by the company AVECIA-NEORESINS, AVALURE UR-405®, AVALURE UR-410®, AVALURE UR-425®, AVALURE UR-450®, SANCURE 875®, SANCURE 861®, SANCURE 878®, SANCURE 2060® by the company GOODRICH, IMPRANIL 85® by the company BAYER, AQUAMERE H-1511® by the company HYDROMER.

As an aqueous dispersion of film-forming polymer, there may also be used the dispersions of polymers resulting from the free-radical polymerization of one or more free-radical monomers inside and/or partly at the surface, of preexisting particles of at least one polymer chosen from polyurethanes, polyureas, polyesters, polyesteramides and/or alkyds. These polymers are generally called hybrid polymers.

According to another aspect of the composition according to the disclosure, the film-forming polymer may be a water-soluble polymer and is therefore present in the aqueous phase of the composition in solubilized form. As examples of film-forming, water-soluble polymers, there may be mentioned:

proteins such as proteins of plant origin, such as wheat or soya bean proteins; proteins of animal origin such as keratins, for example keratin hydrolysates and sulphonic keratins;

anionic, cationic, amphoteric or nonionic polymers of chitin or chitosan;

cellulose polymers such as hydroxyethyl cellulose, hydroxypropyl cellulose, methyl cellulose, ethyl hydroxyethyl cellulose, carboxymethyl cellulose, and quaternized derivatives of cellulose;

acrylic polymers or copolymers such as polyacrylates or polymethacrylates;

vinyl polymers, such as polyvinylpyrrolidones, copolymers of methyl vinyl ether and maleic anhydride, copolymers of vinyl acetate and crotonic acid, copolymers of vinylpyrrolidone and vinyl acetate; copolymers of vinylpyrrolidone and caprolactam; polyvinyl alcohol;

optionally modified polymers of natural origin, such as:

gum arabic, guar gum, xanthan derivatives, karaya gum;

alginates and carrageenans;

glycoaminoglycans, hyaluronic acid and its derivatives;

shellac resin, sandarac gum, dammars, elemis, copals;

deoxyribonucleic acid;

muccopolysaccharides such as chondroitin sulphates.

According to another aspect of the composition according to the disclosure, the film-forming polymer may be present in a liquid fatty phase comprising oils or organic solvents such as those described above. The expression “liquid fatty phase” is understood to mean, in the context of the disclosure, a fatty phase which is liquid at room temperature (25° C.) and atmospheric pressure (760 mmHg, that is 10⁵ Pa), composed of one or more fatty substances which are liquid at room temperature, also called oils, which are generally compatible with each other.

For example, the liquid fatty phase may comprise a volatile oil, optionally in the form of a mixture with a nonvolatile oil, it being possible for the oils to be chosen from the oils cited above.

According to still another aspect of the composition according to the disclosure, the film-forming polymer may be present in the form of surface-stabilized particles dispersed in the liquid fatty phase.

The dispersion of surface-stabilized polymer particles may be manufactured as described in the document EP-A-749747.

The polymer particles are surface-stabilized using a stabilizer which may be a block polymer, a graft polymer and/or a random polymer, alone or in the form of a mixture.

Dispersions of film-forming polymer in the liquid fatty phase, in the presence of stabilizing agents, are for example, described in the documents EP-A-749746, EP-A-923928, EP-A-930060 whose content is incorporated by way of reference into the present application.

The size of the polymer particles in dispersion either in the aqueous phase or in the liquid fatty phase may range from 5 nm to 600 nm, and for instance, from 20 nm to 300 nm.

According to yet another aspect of the composition according to the disclosure, the film-forming polymer may be solubilized in the liquid fatty phase; the film-forming polymer is then said to be a fat-soluble polymer.

By way of example of fat-soluble polymers, there may be mentioned copolymers of vinyl ester (the vinyl group being directly linked to the oxygen atom of the ester group and the vinyl ester having a linear or branched saturated hydrocarbon radical ranging from 1 to 19 carbon atoms, linked to the carbonyl of the ester group) and of at least one other monomer which may be a vinyl ester (different from the vinyl ester already present), α-olefins (having from 8 to 28 carbon atoms), alkyl vinyl ethers (in which the alkyl group comprises from 2 to 18 carbon atoms), or allyl or methallyl esters (having a linear or branched saturated hydrocarbon radical ranging from 1 to 19 carbon atoms, linked to the carbonyl of the ester group).

These copolymers may be crosslinked using crosslinking agents which may be either of the vinyl type, or of the allyl or methallyl type, such as tetraallyloxyethane, divinylbenzene, divinyl octanedioate, divinyl dodecanedioate and divinyl octadecanedioate.

As examples of these copolymers, there may be mentioned the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl propionate/vinyl stearate, allyl dimethyl propionate/vinyl stearate, vinyl propionate/vinyl stearate, crosslinked with 0.2% of divinylbenzene, vinyl dimethyl propionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene crosslinked with 0.2% of divinylbenzene and allyl propionate/allyl stearate crosslinked with 0.2% of divinylbenzene.

As fat-soluble film-forming polymers, there may also be mentioned fat-soluble homopolymers, such as those resulting from the homopolymerization of vinyl esters having from 9 to 22 carbon atoms or of alkyl acrylates or methacrylates, the alkyl radicals having from 10 to 20 carbon atoms.

Such fat-soluble homopolymers may be chosen from polyvinyl stearates, polyvinyl stearates crosslinked using divinylbenzene, diallyl ether and/or diallyl phthalate, polystearyl (meth)acrylate, polyvinyl laurate, and polylauryl (meth)acrylate, it being possible for these poly(meth)acrylates to be crosslinked using ethylene glycol or tetraethylene glycol dimethacrylate.

The fat-soluble copolymers and homopolymers defined above are known and are for instance, described in application FR-A-2232303; they may have a weight-average molecular weight ranging from 2 000 to 500 000, such as from 4 000 to 200 000.

As film-forming, fat-soluble polymers which can be used according to the disclosure, there may also be mentioned polyalkylenes, for example copolymers of C₂-C₂₀ alkenes, such as polybutene, alkyl celluloses with a saturated or unsaturated, linear or branched, C₁ to C₈ alkyl radical such as ethyl cellulose and propyl cellulose, copolymers of vinylpyrrolidone (VP) and for instance, copolymers of vinylpyrrolidone and of a C₂ to C₄₀, such as a C₃ to C₂₀, alkene. By way of example of a VP copolymer which can be used in accordance with the present disclosure, there may be mentioned the VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymer.

The composition according to the disclosure may comprise a film-forming aid which promotes the formation of a film with the film-forming polymer. Such a film-forming agent may be chosen from all the compounds known to persons skilled in the art to be capable of fulfilling the desired function, and for instance, may be chosen from plasticizing agents and coalescing agents.

The composition according to the disclosure may comprise at least one wax.

An aspect of the disclosure is therefore a composition comprising, in a physiologically acceptable medium, substantially rectilinear rigid fibres and at least one wax, the composition having a thixotropic plastic behaviour.

The expression “wax” is understood to mean, within the context of the present disclosure, a lipophilic fatty compound, which is solid at room temperature (25° C.) and atmospheric pressure (760 mmHg, that is 10⁵ Pa), with a reversible solid/liquid change of state, having a melting point greater than 30° C., such as greater than 55° C. and which may range up to 200° C., such as up to 120° C.

By heating the wax to its melting point, it is possible to make it miscible with oils and to form a microscopically homogeneous mixture, but on bringing the temperature of the mixture back to room temperature, recrystallization of the wax in the oils of the mixture is obtained.

The melting point values correspond, according to the disclosure, to the peak of melting measured using a differential scanning calorimeter (DSC), for example the calorimeter sold under the name DSC 30 by the company METLER, with a rise in temperature of 5 or 10° C. per minute.

The waxes, for the purposes of the disclosure, are those generally used in the cosmetic and dermatological fields. There may be mentioned for example, beeswax, lanolin wax, and Chinese waxes; rice wax, Carnauba wax, Candelilla wax, Ouricury wax, cork fibre wax, sugarcane wax, Japan wax, and sumac wax; montan wax, microcrystalline waxes, paraffin waxes, ozokerites, ceresin wax, lignite wax, polyethylene waxes, the waxes obtained by Fischer-Tropsch synthesis, fatty acid esters and glycerides which are solid at 40° C. and for instance at over 55° C.

There may also be mentioned the waxes obtained by catalytic hydrogenation of animal or vegetable oils having linear or branched C₈-C₃₂ fatty chains. Among these, there may be mentioned, for example, hydrogenated jojoba oil, hydrogenated sunflower oil, hydrogenated castor oil, hydrogenated copra oil and hydrogenated lanolin oil.

Silicone waxes and fluorinated waxes may also be mentioned.

A mixture of the waxes described above may be used.

The waxes present in the composition may be dispersed in the form of particles in an aqueous medium. These particles may have a mean size ranging from 50 nm to 50 μm, such as ranging from 50 nm to 10 μm, and for instance, ranging from 50 nm to 3.5 μm.

For example, the wax may be present in the form of a waxes-in-water emulsion, it being possible for the waxes to be in the form of particles having a mean size ranging from 1 μm to 10 μm, for instance ranging from 1 μm to 5 μm.

In another aspect of the composition according to the disclosure, the wax may be present in the form of a wax microdispersion, the wax being in the form of particles that have a mean size less than 1 μm, and for example, ranging from 50 nm to 500 nm. Wax microdispersions are described in the documents EP-A-557196 and EP-A-1048282.

The wax may also have a hardness ranging from 0.05 MPa to 15 MPa, such as ranging from 6 MPa to 15 MPa. The hardness can be determined by measuring the compression force measured at 20° C. using the texturometer sold under the name TA-XT2i by the company RHEO, equipped with a stainless steel cylinder having a diameter of 2 mm, moving at the measuring speed of 0.1 mm/s, and penetrating into the wax to a penetration depth of 0.3 mm. To measure the hardness, the wax is melted at a temperature equal to the melting point of the wax+20° C. The molten wax is poured into a container having a diameter of 30 mm and a depth of 20 mm. The wax is recrystallized at room temperature (25° C.) for 24 hours, and then the wax is stored for at least 1 hour at 20° C. before carrying out the measurement of hardness. The value of the hardness is the measured compacting force divided by the surface area of the texturometer cylinder in contact with the wax.

The wax may be present in the composition according to the disclosure in an amount ranging from 0.1% to 50% by weight, relative to the total weight of the composition, such as from 0.5% to 30% by weight, and for instance from 1% to 20% by weight.

The composition according to the disclosure may comprise at least one fafty compound which is pasty at room temperature. The expression “pasty fatty substance” for the purposes of the disclosure is understood to mean fatty substances having a melting point ranging from 25 to 60° C., such as from 30 to 45° C. and/or a hardness ranging from 0.001 to 0.5 MPa, such as from 0.005 to 0.4 MPa.

The melting point values correspond to the melting point measured using a differential scanning calorimeter (DSC), such as the calorimeter sold under the name DSC 2920 by the company TA Instruments, with a temperature rise of 5 or 10° C. per minute. (The melting point considered is the point corresponding to the temperature of the most endothermic peak of the thermogram).

The hardness can be measured according to the method of penetration of a probe into a sample of compound and for instance, using a texture analyser (for example TA-XT2i from Rheo) equipped with a stainless steel cylinder having a diameter of 2 mm. The hardness is measured at 20° C. at the centre of 5 samples. The cylinder is introduced into each sample at a pre-speed of 1 mm/s and then a measuring speed of 0.1 mm/s, the penetration depth being 0.3 mm. The recorded value of hardness is that of the maximum peak of the applied force.

For example, these fatty substances may be hydrocarbon compounds, optionally of the polymeric type; they may also be chosen from silicone and/or fluorinated compounds; they may also be provided in the form of a mixture of hydrocarbon and/or silicone and/or fluorinated compounds. In the case of a mixture of various pasty fatty substances, pasty hydrocarbon compounds can be predominantly used (comprising mainly carbon and hydrogen atoms and optionally ester groups).

Among the pasty compounds which may be used in the composition according to the disclosure, there may be mentioned lanolins and lanolin derivatives such as acetylated lanolins, oxypropylenated lanolins, andisopropyl lanolate, having a viscosity ranging from 18 to 21 Pa.s, such as from 19 to 20.5 Pa.s, and/or a melting point of 30 to 55° C., and mixtures thereof. It is also possible to use esters of fatty acids and of fatty alcohols, such as those having 20 to 65 carbon atoms (melting point of the order of 20 to 35° C. and/or viscosity at 40° C. ranging from 0.1 to 40 Pa.s) such as triisostearyl or cetyl citrate; arachidyl propionate; vinyl polylaurate; cholesterol esters such as triglycerides of plant origin such as hydrogenated vegetable oils; viscous polyesters such as poly(12-hydroxystearic acid) and mixtures thereof. As triglycerides of plant origin, derivatives of hydrogenated castor oil, such as “THIXINR” from Rheox, may be used.

It is also possible to use silicone pasty fatty substances such as polydimethylsiloxanes (PDMS) having pendent chains of the alkyl or alkoxy type, having from 8 to 24 carbon atoms, and a melting point of 20-55° C., such as stearyl dimethicones, for instance those sold by the company Dow Corning under the trade names DC2503 and DC25514, and mixtures thereof.

The pasty fatty substance may be present in the composition according to the disclosure in an amount ranging from 0 to 60% (such as from 0.01% to 60%) by weight, relative to the total weight of the composition, for instance ranging from 0.5 to 45% by weight, and for further example ranging from 2% to 30% by weight, in the composition.

The composition according to the disclosure may contain emulsifying surfactants present, for example, in a proportion ranging from 2 to 30% by weight, relative to the total weight of the composition, such as from 5% to 15% by weight, relative to the total weight of the composition. These surfactants may be chosen from anionic, nonionic and amphoteric surfactants. Reference may be made to the document “Encyclopedia of Chemical Technology, KIRK-OTHMER”, volume 22, p. 333-432, 3^{rd} edition, 1979, WILEY, for the definition of the properties and functions (emulsifier) of the surfactants, for example, p. 347-377 of this reference, for anionic and nonionic surfactants.

Mention may be made of the following non-limiting examples of surfactants that may be used in the composition according to the disclosure:

from nonionic surfactants: fatty acids, fatty alcohols, polyethoxylated and polyglycerolated fatty alcohols such as polyethoxylated stearyl and cetylstearyl alcohols, esters of fatty acid and of sucrose, esters of alkyl glucose, such as polyoxyethylenated fatty esters of C₁-C₆ alkyl glucose, sorbitol or glycerol mono-, di-, tri- or sesquioleates or stearates, glycerol laureates, polyethylene glycol laurates; alkyl and alkoxy dimethicone copolyols with an alkyl or alkoxy chain which is pendent or at the end of the silicone backbone having for example 6 to 22 carbon atoms, polyethylene glycol fatty acid esters (polyethylene glycol monostearate or monolaurate); polyoxyethylenated sorbitol fatty acid esters (stearate, oleate); polyoxyethylenated alkyl (lauryl, cetyl, stearyl, octyl) ethers, dimethicone copolyols, and mixtures thereof.

from anionic surfactants: C₁₆-C₃₀ fatty acids neutralized with amines, aqueous ammonia or alkali-metal salts, and mixtures thereof.

For example, surfactants may be used which allow the production of an oil-in-water or wax-in-water emulsion.

The composition may comprise additional short fibres, different from the rigid fibres described above and having, for example, a length of less than 0.8 mm, such as ranging from 0.1 mm to 0.5 mm.

The additional short fibres may be chosen from silk fibres, cotton fibres, wool fibres, flax fibres, cellulose fibres extracted, for example, from wood, from vegetables, and from algae, polyamide fibres (Nylon®), modified cellulose fibres (rayon, viscose, acetate, such as rayon acetate), acrylic, such as polymethyl methacrylate or poly-2-hydroxyethyl methacrylate, fibres, polyolefin, such as polyethylene or polypropylene, fibres, Teflon® fibres, insoluble collagen fibres, polyester fibres, polyvinyl chloride fibres, polyvinylidene chloride fibres, polyvinyl alcohol fibres, polyacrylonitrile fibres, chitosan fibres, polyurethane fibres, and polyethylene phthalate fibres.

The composition according to the disclosure may also comprise at least one colouring substance such as pulverulent colouring substances, fat-soluble colorants, and water-soluble colorants. The at least one colouring substance may be present in an amount ranging from 0.01% to 30% by weight, relative to the total weight of the composition.

The pulverulent colouring substances may be chosen from pigments and pearlescent agents.

The pigments may be white and/or coloured, inorganic and/or organic, coated or otherwise. There may be mentioned, among the inorganic pigments, titanium dioxide, optionally surface-treated, zirconium, zinc or cerium oxides, as well as iron or chromium oxides, manganese violet, ultramarine blue, chromium hydrate and ferric blue. Among the organic pigments, there may be mentioned carbon black, pigments of the D & C type, and lacquers based on carmine, barium, strontium, calcium or aluminium.

The pearlescent agents may be chosen from white pearlescent pigments such as mica coated with titanium or bismuth oxychloride, coloured pearlescent pigments such as mica-titanium with iron oxides, mica-titanium with, for example, ferric blue or chromium oxide, mica-titanium with an organic pigment of the abovementioned type as well as pearlescent pigments based on bismuth oxychloride.

The fat-soluble colorants may be chosen from, for example, Sudan red, D&C Red 17, D&C Green 6, β-carotene, soya-bean oil, Sudan brown, D&C Yellow 11, D&C Violet 2, D&C Orange 5, quinoline yellow, and annatto. The water-soluble colorants may be chosen from, for example, sugarbeet juice and methylene blue.

The composition according to the disclosure may additionally comprise fillers. The expression fillers should be understood to mean particles of any form, which are colourless or white, inorganic or synthetic, insoluble in the medium of the composition regardless of the temperature at which the composition is produced. The fillers serve for example to modify the rheology or the texture of the composition.

The fillers may be inorganic and/or organic, of any form, platelet, spherical and/or oblong, regardless of the crystallographic form (for example sheet, cubic, hexagonal, orthorhombic, and the like). There may be mentioned talc, mica, silica, kaolin, powders of polyamide (Nylon®) (Orgasol® from Atochem), of poly-β-alanine and of polyethylene, powders of tetrafluoroethylene polymers (Teflon®), lauroyllysine, starch, boron nitride, polymeric hollow microspheres such as those of polyvinylidene chloride/acrylonitrile such as Expancel® (Nobel Industrie), acrylic acid copolymers (Polytrap® from the company Dow Corning) and microbeads of silicone resin (Tospearls® from Toshiba, for example), particles of elastomeric polyorganosiloxanes, polymethyl methacrylate microbeads, precipitated calcium carbonate, magnesium carbonate and hydrocarbonate, hydroxyapatite, hollow microspheres of silica (Silica Beads® from Maprecos), glass and/or ceramic microcapsules, metal soaps derived from organic carboxylic acids having from 8 to 22 carbon atoms, such as from 12 to 18 carbon atoms, for example zinc, magnesium or lithium stearate, zinc laurate, magnesium myristate.

The fillers may be present in an amount ranging from 0.01 to 30% by weight, such as from 0.5% to 15% by weight.

The composition of the disclosure may additionally comprise at least one of any additive customarily used in cosmetics such as antioxidants, preservatives, perfumes, neutralizing agents, cosmetic and dermatological active agents such as, for example, emollients, moisturizers, vitamins, and sunscreens. These additives may be present in the composition in an amount ranging from 0.01 to 20% of the total weight of the composition, such as ranging from 0.01 to 10%.

Of course, persons of ordinary skill in the art will be careful to choose the possible additional additives and/or their quantity such that the beneficial properties of the composition according to the disclosure are not, or not substantially, impaired by the addition envisaged.

The composition according to the disclosure may be produced by the known methods generally used in the cosmetic and/or dermatological fields.

The invention is illustrated in greater detail, but in a non-limiting manner, in the following examples.

### EXAMPLE 1

A mascara having the following composition was prepared:

| | |
|---|---|
| polyimide-amide fibres 2 mm long sold under | 0.63 g |
| the name KERMEL TECH by the company Rhodia | |
| saponite (Veegum DGT by the company | 2.63 g |
| Vanderbilt) | |
| associative polyurethane (Ser Ad FX 1100 | 2 g |
| from the company Servo) | |
| sulphopolyester (AQ 55 S from the company | 3 g |
| Eastman Chemical) | |
| beeswax | 8 g |
| polyvinylpyrrolidone/1-eicosene copolymer | 3 g |
| (Antaron V 220F from the company ISP) | |
| microbead of methyl methacrylate/ethylene | 4 g |
| glycol dimethacrylate copolymer | |
| polyamide fibres 0.3 mm long and 0.9 Dtex | 2 g |
| from the company Paul Bonte | |
| propylene glycol | 8 g |
| black iron oxide | 8 g |
| preservatives qs | |
| water qs | 100 g |

This mascara had the following rheological profile:

initial consistency Gᵢ* equal to 10 000 Pa

initial elasticity δᵢ equal to 26°

yield point c equal to 100 Pa;

resumption of thixotropy, after a continuous shearing of 1 000 s⁻¹, for one minute equal to 32% after 1 minute of rest and equal to 84.5% after 30 minutes of rest.

The rigid polyimide-amide fibres were homogeneously dispersed in the mascara. The mascara was easy to apply to the eyelashes and made it possible to obtain an optimum lengthening of the eyelashes, the rigid fibres being fixed in the optimum continuation of the eyelashes.

### EXAMPLE 2

A mascara having the following composition was prepared:

| | |
|---|---|
| polyimide-amide fibres 2 mm long sold under | 1 g |
| the name KERMEL TECH by the company Rhodia | |
| hectorite modified with distearyldimethyl- | 11 g |
| ammonium chloride sold under the name | |
| Bentone 38V by the company Elementis | |
| propylene carbonate | 2 g |
| vinyl acetate/allyl stearate (65/35) | 4.5 g |
| copolymer (Mexomere PQ from CHIMEX) | |
| polyvinyl laurate (Mexomere PP from CHIMEX) | 1.5 g |
| black iron oxide | 7 g |
| preservative qs | |
| isododecane qs | 100 g |

The mascara obtained had a thixotropic plastic behaviour; it was easy to apply to the eyelashes and conferred on the eyelashes a lengthening effect which was homogeneous and situated in the continuation of the eyelashes.

### EXAMPLE 3

A mascara having the following composition was prepared:

| | |
|---|---|
| polyimide-amide fibres 2 mm long sold under | 1 g |
| the name KERMEL TECH by the company Rhodia | |
| hectorite modified with distearyldimethyl- | 11 g |
| ammonium chloride sold under the name | |
| Bentone 38V by the company Elementis | |
| propylene carbonate | 2 g |
| carnauba wax | 8 g |
| black iron oxide | 7 g |
| preservative qs | |
| isododecane qs | 100 g |

The mascara obtained had a thixotropic plastic behaviour; it was easy to apply to the eyelashes and conferred on the eyelashes a lengthening effect which was homogeneous and situated in the continuation of the eyelashes.

What is claimed is:

1. A composition comprising, in a physiologically acceptable medium, rigid fibres and at least one compound chosen from film-forming polymers and waxes, wherein the composition has a thixotropic plastic behaviour.

2. The composition according to claim 1, wherein at least 50% of the fibers, in numerical terms, are such that the angle formed between the tangent to the longitudinal central axis of the fibre at an end of the fibre and the line joining the said end to the point on the longitudinal central axis of the fibre corresponding to half the length of the fibre is less than 15°,
and the angle formed between the tangent to the longitudinal central axis of the fibre at a point situated halfway along the fibre and the line joining one of the ends to the point on the longitudinal central axis of the fibre corresponding to half the length of the fibre is less than or equal to 15° for an identical length of fibre ranging from 0.8 mm to 5 mm.3.The composition according to claim 2, wherein at least 75% of the fibers, in numerical terms, are such that the angle formed between the tangent to the longitudinal central axis of the fibre at an end of the fibre and the line joining the said end to the point on the longitudinal central axis of the fibre corresponding to half the length of the fibre is less than 15°.

3. The composition according to claim 2, wherein at least 75% of the fibers, in numerical terms, are such that the angle formed between the tangent to the longitudinal central axis of the fibre at an end of the fibre and the line joining the said end to the point on the longitudinal central axis of the fibre corresponding to half the length of the fibre is less than 15°.

4. The composition according to claim 3, wherein at least 90% of the fibers, in numerical terms, are such that the angle formed between the tangent to the longitudinal central axis of the fibre at an end of the fibre and the line joining the said end to the point on the longitudinal central axis of the fibre corresponding to half the length of the fibre is less than 15°.

5. The composition according to claim 2, wherein the angle formed between the tangent to the longitudinal central axis of the fibre at a point situated halfway along the fibre and the line joining one of the ends to the point on the longitudinal central axis of the fibre corresponding to half the length of the fibre is less than or equal to 15° for an identical length of fibre ranging from 1 mm to 4 mm.

6. The composition according to claim 5, wherein the angle formed between the tangent to the longitudinal central axis of the fibre at a point situated halfway along the fibre and the line joining one of the ends to the point on the longitudinal central axis of the fibre corresponding to half the length of the fibre is less than or equal to 15° for an identical length of fibre ranging from 1 mm to 3 mm.

7. The composition according to claim 6, wherein the angle formed between the tangent to the longitudinal central axis of the fibre at a point situated halfway along the fibre and the line joining one of the ends to the point on the longitudinal central axis of the fibre corresponding to half the length of the fibre is less than or equal to 15° for an identical length of fibre of 2 mm.

8. The composition according to claim 2, wherein the said angle is less than or equal to 10°.

9. The composition according to claim 8, wherein the said angle is less than or equal to 5°.

10. The composition according to claim 1, wherein the substantially rectilinear rigid fibres have a length (L) ranging from 0.8 mm to 5 mm,

11. The composition according to claim 10, wherein the substantially rectilinear rigid fibres have a length (L) ranging from 1 mm to 4 mm.

12. The composition according to claim 11, wherein the substantially rectilinear rigid fibres have a length (L) ranging from 1 mm to 3 mm.

13. The composition according to claim 1, wherein the substantially rectilinear rigid fibres have a section contained in a circle having a diameter (D) ranging from 2 nm to 500 μm.

14. The composition according to claim 13, wherein the substantially rectilinear rigid fibres have a section contained in a circle having a diameter (D) ranging from 100 nm to 100 μm.

15. The composition according to claim 14, wherein the substantially rectilinear rigid fibres have a section contained in a circle having a diameter (D) ranging from 1 μm to 50 μm.

16. The composition according to claim 1, wherein the substantially rectilinear rigid fibres have an aspect ratio (L/D) ranging from 3.5 to 2,500.

17. The composition according to claim 16, wherein the substantially rectilinear rigid fibres have an aspect ratio (L/D) ranging from 5 to 500.

18. The composition according to claim 17, wherein the substantially rectilinear rigid fibres have an aspect ratio (L/D) ranging from 5 to 150.

19. The composition according to claim 1, wherein the substantially rectilinear rigid fibres have a linear density ranging from 0.15 to 30 denier.

20. The composition according to claim 19, wherein the substantially rectilinear rigid fibres have a linear density ranging from 0.18 to 18 denier.

21. The composition according to claim 1, wherein the substantially rectilinear rigid fibres are fibres of a synthetic polymer chosen from polyesters, polyurethanes, acrylic polymers, polyolefins, and polyamides.

22. The composition according to claim 1, wherein the substantially rectilineaer rigid fibres do not comprise several alternate layers of polymers having different refractive indices.

23. The composition according to claim 1, wherein the substantially rectilinear rigid fibres are aromatic polyimide-amide fibres.

24. The composition according to claim 1, wherein the substantially rectilinear rigid fibres are aromatic polyimide-amide fibres chosen from aromatic polyimide-amides comprising a repeating unit of formula (I): 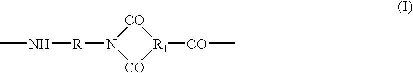
and optionally additionally comprising at least one repeating unit chosen from formulae (II), (III), and (IV):
—NH—R—NH—CO—R₂—CO— (II) 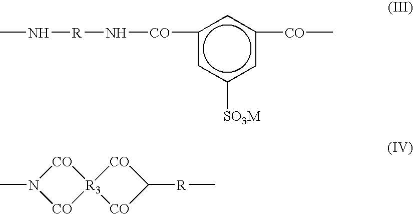
wherein R and R₂, which may be the same or different, are chosen from divalent aromatic groups, R₁ is chosen from trivalent aromatic groups, R₃ is chosen from tetravalent aromatic groups, and M is chosen from alkali metals and alkaline-earth metals.

25. The composition according to claim 24, wherein R₁ is chosen from:

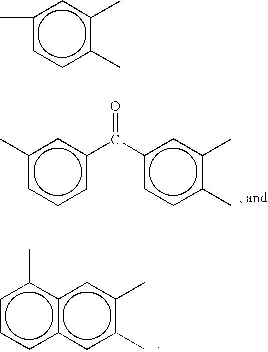

26. The composition according to claim 24, wherein R is chosen from:

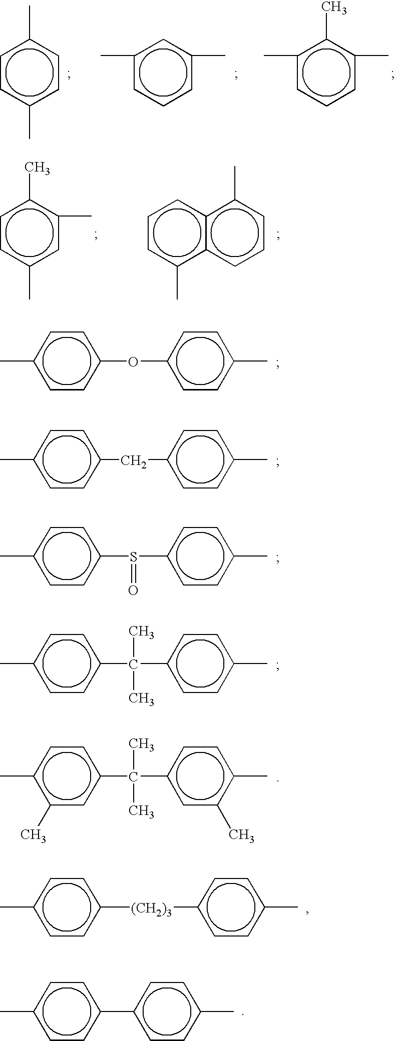

27. The composition according to claim 24, wherein R₂ is chosen from:

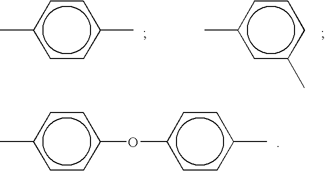

28. The composition according to claim 24, wherein R₃ is chosen from:

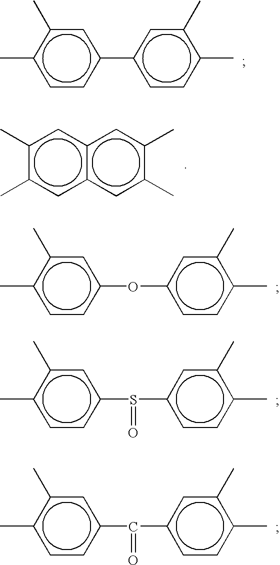

29. The composition according to claim 24, wherein the polyimide-amide is obtained by polymerization of tolylene diisocyanate and trimellitic anhydride, and comprises repeating units of formula:

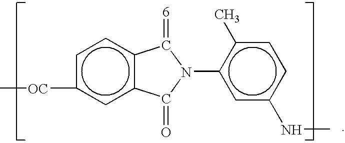

30. The composition according to claim 1, wherein the substantially rectilinear rigid fibres are present in an amount ranging from 0.01% to 10% by weight, relative to the total weight of the composition.

31. The composition according to claim 30, wherein the substantially rectilinear rigid fibres are present in an amount ranging from 0.1% to 5% by weight, relative to the total weight of the composition.

32. The composition according to claim 31, wherein the substantially rectilinear rigid fibres are present in an amount ranging from 0.3% to 3% by weight, relative to the total weight of the composition.

33. The composition according to claim 1, wherein said composition has an initial consistency G*ᵢ ranging from 1×10² Pa to 1×10⁵ Pa, measured under a sinusoidal stress at a frequency of 1 Hz.

34. The composition according to claim 33, wherein said composition has an initial consistency G*ᵢ ranging from 5×10² Pa to 5×10⁴ Pa, measured under a sinusoidal stress at a frequency of 1 Hz.

35. The composition according to claim 34, wherein said composition has an initial consistency G*ᵢ ranging from 6×10² Pa to 9×10³ Pa, measured under a sinusoidal stress at a frequency of 1 Hz.

36. The composition according to claim 1, wherein said composition has an initial elasticity δᵢ ranging from 1° to 45°.

37. The composition according to claim 36, wherein said composition has an initial elasticity δᵢ ranging from 10° to 35°.

38. The composition according to claim 1, wherein said composition has a yield point τ_{C} ranging from 10 Pa to 3 500 Pa.

39. The composition according to claim 1, wherein said composition has a yield point τ_{C} ranging from 20 Pa to 1 000 Pa.

40. The composition according to claim 1, further comprising at least one thixotropic thickening agent.

41. The composition according to claim 1, wherein said composition comprises an aqueous medium.

42. The composition according to claim 1, wherein said composition comprises water and optionally at least one hydrophilic organic solvent.

43. The composition according to claim 42, wherein the at least one hydrophilic organic solvent is chosen from monoalcohols having from 2 to 5 carbon atoms, polyols having from 2 to 8 carbon atoms, C₃-C₄ ketones, and C₂-C₄ aldehydes.

44. The composition according to claim 42, wherein the water or the mixture of water and the at least one hydrophilic organic solvent is present in an amount ranging from 0.1% to 90% by weight, relative to the total weight of the composition.

45. The composition according to claim 44, wherein the water or the mixture of water and at least one hydrophilic organic solvent is present in an amount ranging from 0.1% to 60% by weight.

46. The composition according to claim 40, wherein the at least one thixotropic thickening agent is a thixotropic thickener for aqueous mediums chosen from hydrophilic clay, carrageenan gum, and hydrophilic pyrogenic silica.

47. The composition according to claim 46, wherein the hydrophilic clay is a clay chosen from smectites, vermiculites, stevensite, and chlorites.

48. The composition according to claim 46, wherein the hydrophilic clay is chosen from montmorillonites, hectorites, bentonites, beidellite, and saponites.

49. The composition according to claim 1, wherein said composition comprises a liquid fatty phase.

50. The composition according to claim 49, wherein the liquid fatty phase comprises at least one fatty substance chosen from oils and organic solvents.

51. The composition according to claim 50, wherein said composition comprises at least one oil chosen from oils of mineral, animal, plant, and synthetic origin, carbonaceous oils, hydrocarbonaceous oils, fluorinated oils, and silicone oils.

52. The composition according to claim 49, wherein the liquid fatty phase is present in an amount ranging from 0.1% to 98% by weight, relative to the total weight of the composition.

53. The composition according to claim 52, wherein the liquid fatty phase is present in an amount ranging from 1 to 80% by weight, relative to the total weight of the composition.

54. The composition according to claim 50, wherein said composition comprises at least one volatile oil and/or at least one volatile organic solvent.

55. The composition according to claim 54, wherein the at least one volatile oil is chosen from octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and branched C₈-C₁₆ alkanes.

56. The composition according to claim 54, wherein the at least one volatile oil is present in an amount ranging from 0.1% to 98% by weight, relative to the total weight of the composition.

57. The composition according to claim 56, wherein the at least one volatile oil is present in an amount ranging from 1% to 65% by weight, relative to the total weight of the composition.

58. The composition according to claim 50, wherein said composition comprises at least one nonvolatile oil.

59. The composition according to claim 58, wherein the at least one nonvolatile oil is present in an amount ranging from 0.1% to 80% by weight, relative to the total weight of the composition.

60. The composition according to claim 59, wherein the at least one nonvolatile oil is present in an amount ranging from 0.1% to 50% by weight, relative to the total weight of the composition.

61. The composition according to claim 60, wherein the at least one nonvolatile oil is present in an amount ranging from 0.1% to 20% by weight, relative to the total weight of the composition.

62. The composition according to claim 40, wherein the at least one thixotropic thickening agent is chosen from thixotropic thickeners for an oily medium.

63. The composition according to claim 62, wherein the at least one thixotropic thickening agent for the oily medium is chosen from organophilic clays, hydrophobic pyrogenic silicas, and elastomeric organopolysiloxanes.

64. The composition according to claim 40, wherein the at least one thixotropic thickening agent is present in an amount ranging from 0.5% to 15% by weight, relative to the total weight of the composition.

65. The composition according to claim 64, wherein the at least one thixotropic thickening agent is present in an amount ranging from 1% to 15% by weight, relative to the total weight of the composition.

66. The composition according to claim 65, wherein the at least one thixotropic thickening agent is present in an amount ranging from 2% to 10% by weight, relative to the total weight of the composition.

67. The composition according to claim 66, wherein the at least one thixotropic thickening agent is present in an amount ranging from 2% to 8% by weight, relative to the total weight of the composition.

68. The composition according to claim 40, further comprising at least one additional thickening agent.

69. The composition according to claim 40, wherein the at least one additional thickening agent is a hydrophilic thickening agent.

70. The composition according to claim 69, wherein the at least one additional hydrophilic thickening agent is an associative polyurethane.

71. The composition according to claim 68, wherein the at least one additional thickening agent is a lipophilic thickening agent.

72. The composition according to claim 68, wherein the at least one additional thickening agent is present in an amount ranging from 0.1% to 5% by weight, relative to the total weight of the composition.

73. The composition according to claim 72, wherein the at least one additional thickening agent is present in an amount ranging from 0.1% to 3% by weight, relative to the total weight of the composition.

74. The composition according to claim 1, wherein the at least one film-forming polymer is chosen from vinyl polymers, acrylic polymers, polyurethanes, polyesters, polyamides, polyureas, and cellulosic polymers.

75. The composition according to claim 1, wherein the at least one film-forming polymer is present in the form of particles in aqueous dispersion.

76. The composition according to claim 1, wherein the at least one film-forming polymer is present in a polymer dry matter content ranging from 0.1% to 60% by weight, relative to the total weight of the composition.

77. The composition according to claim 76, wherein the at least one film-forming polymer is present in a polymer dry matter content ranging from 0.5% to 40% by weight, relative to the total weight of the composition.

78. The composition according to claim 77, wherein the at least one film-forming polymer is present in a polymer dry matter content ranging from 1% to 30% by weight, relative to the total weight of the composition.

79. The composition according to claim 1, wherein the wax has a melting point ranging from 30° C. to 120° C.

80. The composition according to claim 1, wherein the wax is chosen from beeswax, lanolin wax, Chinese waxes; rice wax, Carnauba wax, Candelilla wax, Ouricury wax, cork fibre wax, sugarcane wax, Japan wax, sumac wax; montan wax, microcrystalline waxes, paraffin waxes, ozokerites, ceresin wax, lignite wax, polyethylene waxes, the waxes obtained by Fischer-Tropsch synthesis, fatty acid esters, glycerides which are solid at 40° C., waxes obtained by catalytic hydrogenation of animal or vegetable oils having linear and/or branched C₈-C₃₂ fatty chains; silicone waxes, and fluorinated waxes.

81. The composition according to claim 1, wherein the wax has a hardness ranging from 0.05 MPa to 15 MPa.

82. The composition according to claim 1, wherein the wax is present in an amount ranging from 0.1% to 50% by weight, relative to the total weight of the composition.

83. The composition according to claim 82, wherein the wax is present in an amount ranging from 0.5% to 30% by weight, relative to the total weight of the composition.

84. The composition according to claim 83, wherein the wax is present in an amount ranging from 1% to 20% by weight, relative to the total weight of the composition.

85. The composition according to claim 1, further comprising at least one pasty fatty substance.

86. The composition according to claim 1, further comprising at least one surfactant.

87. The composition according to claim 1, further comprising additional short fibres having a length of less than 0.8 mm, wherein said additional short fibres are different from the substantially rectilinear rigid fibres.

88. The composition according to claim 87, wherein the length of the additional short fibres ranges from 0.1 mm to 0.5 mm.

89. The composition according to claim 1, further comprising at least one colouring substance.

90. The composition according to claim 89, wherein the at least one colouring substance is chosen from pigments, pearlescent agents, fat-soluble colorants, and water-soluble colorants.

91. The composition according to claim 89, wherein the at least one colouring substance is present in an amount ranging from 0.01% to 30% by weight, relative to the total weight of the composition.

92. The composition according to claim 1, further comprising at least one filler.

93. The composition according to claim 1, further comprising at least one cosmetic additive chosen from antioxidants, preservatives, perfumes, neutralizing agents, emollients, moisturizers, vitamins, sunscreens, plasticizing agents, and coalescing agents.

94. A composition comprising, in a physiologically acceptable medium, substantially rectilinear rigid fibres and at least one compound chosen from film-forming polymers and waxes, wherein the composition has a thixotropic plastic behaviour,
and wherein said composition is in a form chosen from a composition for coating the eyelashes, a product for the eyebrows, an eyeliner, a product for the lips, a blusher, an eye shadow, a foundation, a make-up product for the body, a concealer, a nail varnish, and a care product for the skin.

95. A composition comprising, in a physiologically acceptable medium, substantially rectilinear rigid fibres and at least one compound chosen from film-forming polymers and waxes, wherein the composition has a thixotropic plastic behaviour, and
wherein said composition is in a form chosen from a care composition for keratinous fibres and a make-up composition for keratinous fibres.

96. A composition comprising, in a physiologically acceptable medium, substantially rectilinear rigid fibres and at least one compound chosen from film-forming polymers and waxes, wherein the composition has a thixotropic plastic behaviour, and
wherein the composition is a mascara.

97. A cosmetic method for making up and caring for keratinous materials comprising applying to keratinous materials a composition comprising, in a physiologically acceptable medium, substantially rectilinear rigid fibres and at least one compound chosen from film-forming polymers and waxes, wherein the composition has a thixotropic plastic behaviour.

98. The cosmetic method according to claim 97, wherein the keratinous materials are eyelashes.

99. A method for obtaining a homogenous deposit on keratinous materials, comprising
applying to the keratinous materials a composition comprising, in a physiologically acceptable medium, substantially rectilinear rigid fibres and at least one compound chosen from film-forming polymers and waxes, wherein the composition has a thixotropic plastic behaviour.

100. A method for obtaining a lengthening effect of eyelashes comprising,
applying to the eyelashes a composition comprising, in a physiologically acceptable medium,
substantially rectilinear rigid fibres and at least one compound chosen from film-forming polymers and waxes, wherein the composition has a thixotropic plastic behaviour.
